(19) United States
(12) Patent Application Publication
(10) Pub. No.: US 2023/0316639 A1
(43) Pub. Date: Oct. 5, 2023

(54) SYSTEMS AND METHODS FOR ENHANCING MEDICAL IMAGES

(71) Applicant: Activ Surgical, Inc., Boston, MA (US)

(72) Inventors: Vasiliy Buharin, Boston, MA (US); Roman Stolyarov, Boston, MA (US); John Galeotti, Boston, MA (US)

(21) Appl. No.: 17/938,614

(22) Filed: Oct. 6, 2022

Related U.S. Application Data
(63) Continuation of application No. PCT/US2021/027710, filed on Apr. 16, 2021.
(60) Provisional application No. 63/011,740, filed on Apr. 17, 2020.

(51) Int. Cl. G06T 15/60; G06T 15/50; G06T 15/06; G06T 7/50; G06V 10/24; G06V 10/25; A61B 90/00

(52) U.S. Cl. CPC G06T 15/60; A61B 90/361; G06T 7/50; G06T 15/06; G06T 15/506; G06V 10/242; G06V 10/25; G06T 2207/10081; G06T 2207/10088

(57) ABSTRACT

The present disclosure provides methods for enhancing depth perception. The method may comprise: using a scope and an imaging device to obtain an image and a depth map of a surgical scene, identifying a region of interest within the image or depth map, simulating a virtual light model comprising a plurality of virtual light sources configured to generate one or more virtual light beams, rotating the depth map and the image to align a plurality of pixels with the one or more virtual light beams, and using an image processing algorithm to generate one or more virtual shadows for one or more portions of the region of interest based in part on the rotated image and the rotated depth map, thereby enhancing depth perception within the image of the surgical scene to aid the surgical procedure.

### CROSS-REFERENCE

This application is a continuation of International Application No. PCT/US2021/027710, filed on Apr. 16, 2021, which claims priority to U.S. Provisional Application No. 63/011,740, filed on Apr. 17, 2020, each of which is incorporated herein by reference in its entirety for all purposes.

### BACKGROUND

Medical imaging technology may be used to capture images or video data of internal anatomical features of a subject or patient during medical or surgical procedures. The images or video data captured may be processed and manipulated to provide surgeons and medical operators with an enhanced visualization of internal structures or processes within a patient or subject. Conventional medical imaging systems available today may be configured to provide images that do not contain shadows, since such shadows may occlude certain regions of interest. However, such systems may also limit the depth perception of an operator who is viewing a region of interest.

### SUMMARY

Recognized herein are various limitations with medical imaging systems currently available. The present disclosure provides systems and methods that can address existing shortcomings or deficiencies of conventional medical imaging systems. The systems and methods disclosed herein may be used to enhance medical imaging by selectively generating virtual shadows that can provide an operator with enhanced depth perception. Further, the systems and methods disclosed herein may be implemented to selectively position and reposition one or more virtual shadows, thereby allowing medical operators to visualize anatomical structures without having one or more virtual shadows occlude a region of interest and without losing monocular cues that can aid in depth perception. The systems and methods disclosed herein may also be implemented to adjust the shading in medical images to augment depth perception. In some cases, the systems and methods disclosed herein may be used for dynamic, real-time augmented reality image overlays for deformable tissue regions undergoing physical deformations. As such, the systems and methods disclosed herein can provide medical operators with additional visual information that can enhance the medical operators’ depth perception and inform or guide them during a surgical procedure.

In an aspect, the present disclosure provides a method for enhancing depth perception to aid a surgical procedure. The method may comprise: (a) using a scope and an imaging device to obtain (i) an image of a surgical scene and (ii) a depth map associated with the image. The scope may be optically coupled to the imaging device. The method may further comprise (b) identifying a region of interest within the image or the depth map. The region of interest may comprise a plurality of pixels. The method may further comprise (c) simulating a virtual light model. The virtual light model may comprise a plurality of virtual light sources configured to generate one or more virtual light beams that intersect the image of the surgical scene. The method may further comprise (d) rotating the depth map and the image of the surgical scene by a rotational angle to align the plurality of pixels with the one or more virtual light beams. The rotational angle may be computed based in part on an angle formed between (i) a reference line comprising two or more pixels within the region of interest and (ii) a projection of the one or more virtual light beams onto a reference plane containing the image of the surgical scene. The method may further comprise (e) using an image processing algorithm to generate one or more virtual shadows for one or more portions of the region of interest based in part on the rotated image and the rotated depth map, thereby enhancing depth perception within the image of the surgical scene to aid the surgical procedure. In some embodiments, the depth map may comprise a two-dimensional (2D) data array or data structure.

In another aspect, the present disclosure provides a method for enhancing depth perception to aid a surgical procedure. The method may comprise (a) obtaining an image of a surgical scene and a depth map associated with the image, wherein the image does not comprise a pre-operative image; and (b) using an image processing algorithm to directly generate, based in part on the image and the depth map, one or more virtual shadows for enhancing depth perception in the image, without using or requiring computation of a three-dimensional (3D) representation of the surgical scene. In some embodiments, the image may not comprise a superimposed image. In some embodiments, the pre-operative image may comprise a pre-operative scan selected from the group consisting of a computed tomographic (CT) scan, a magnetic resonance imaging (MRI) scan, and an ultrasonography scan. In some embodiments, the three-dimensional (3D) representation may comprise a 3D data array or data structure, a 3D volume, a point cloud or a mesh associated with the surgical scene or an anatomical feature within the surgical scene.

In some embodiments, the image processing algorithm may be configured to simulate a virtual light model comprising a plurality of virtual light sources. In some embodiments, the plurality of virtual light sources may be configured to generate one or more virtual light beams that intersect the image of the surgical scene to generate one or more virtual shadows within a portion of the image.

In some embodiments, the image processing algorithm may be configured to implement a shadow mapping algorithm to generate the one or more virtual shadows. In some embodiments, the shadow mapping algorithm may be configured to generate the one or more virtual shadows using a modified image of the surgical scene, which modified image may be derived by rotating the image of the surgical scene to align two or more pixels of the image with one or more virtual light beams.

In some embodiments, the shadow mapping algorithm may be configured to use the modified image to compute shadow map values for an array of pixels arranged in one or more pixel columns that are aligned with the one or more virtual light beams, thereby reducing an amount of computation required to generate shadow map values for pixels that are not aligned with the one or more virtual light beams.

In some embodiments, the shadow mapping algorithm may be configured to use the modified image to process two or more columns of pixels in parallel when computing shadow map values for a plurality of pixels within the two or more columns of pixels, thereby improving an efficiency of the shadow mapping algorithm.

In some embodiments, the shadow mapping algorithm may be configured to generate the one or more virtual shadows in part using ray tracing.

In some embodiments, the one or more virtual light beams may be parallel. In some embodiments, the one or more virtual light beams are generated using a plurality of virtual light sources simulated within or near the surgical scene. In some embodiments, the plurality of virtual light sources may be repositionable to generate the one or more virtual shadows in different regions of interest.

In another aspect, the present disclosure provides a method for enhancing depth perception to aid a surgical procedure. The method may comprise: (a) using a scope and an imaging device to obtain (i) an image of a surgical scene and (ii) a depth map associated with the image, wherein the scope is optically coupled to the imaging device; (b) identifying a region of interest within the image or the depth map, wherein the region of interest comprises a plurality of pixels; (c) simulating a virtual light model, wherein the virtual light model comprises a plurality of virtual light sources configured to generate one or more virtual light beams that intersect the image of the surgical scene; (d) rotating the depth map and the image of the surgical scene by a rotational angle to align the plurality of pixels with the one or more virtual light beams; and (e) using an image processing algorithm to generate one or more virtual shadows for one or more portions of the region of interest based in part on the rotated image and the rotated depth map, thereby enhancing depth perception within the image of the surgical scene to aid the surgical procedure.

In some embodiments, the rotational angle may be computed based at least in part on an angle formed between (i) a reference line comprising two or more pixels within the region of interest and (ii) a projection of the one or more virtual light beams onto a reference plane containing the image of the surgical scene.

In some embodiments, the image processing algorithm may be configured to implement a shadow mapping algorithm to generate the one or more virtual shadows. In some embodiments, the shadow mapping algorithm may be configured to compute one or more shadow maps in part based on (i) a position and an orientation of the one or more virtual light sources relative to a portion of the region of interest and (ii) a comparison of depth values for two or more pixels within the portion of the region of interest. In some embodiments, the two or more pixels may comprise two or more adjacent pixels within a row or column of pixels.

In some embodiments, a virtual shadow may be drawn for a first pixel when the first pixel has a greater depth map value than a second pixel that is positioned between the first pixel and the virtual light source.

In some embodiments, the shadow mapping algorithm may be configured to use the rotated image to compute shadow map values for an array of pixels arranged in one or more pixel columns that are aligned with the one or more virtual light beams, thereby reducing an amount of computation required to generate shadow map values for pixels that are not aligned with the one or more virtual light beams.

In some embodiments, the shadow mapping algorithm may be configured to use the rotated image to process two or more columns of pixels in parallel when computing shadow map values for a plurality of pixels within the two or more columns of pixels, thereby improving an efficiency of the shadow mapping algorithm.

In some embodiments, the shadow mapping algorithm may be configured to generate the one or more virtual shadows in part using ray tracing.

In some embodiments, the one or more virtual light beams may be parallel.

In some embodiments, the scope may comprise a laparoscope, an endoscope, a borescope, a videoscope, or a fiberscope.

In some embodiments, the plurality of virtual light sources may be repositionable to generate the one or more virtual shadows in a different region of interest.

In some embodiments, the rotational angle may be greater than 0 degrees and less than or equal to 360 degrees.

In another aspect, the present disclosure provides a system for enhancing depth perception to aid a surgical procedure. The system may comprise: (a) a scope that is insertable into a body of a subject; (b) an imaging device optically coupled to the scope, wherein the imaging device is configured to (i) obtain one or more two-dimensional (2D) images of a surgical scene within the body of the subject and (ii) measure depth information or compute a topology of the surgical scene; and (c) an image processing module configured to use the one or more two-dimensional images and at least one of (i) the depth information or (ii) the topology of the surgical scene to directly generate one or more virtual shadows in the one or more two-dimensional images without using or requiring computation of a three-dimensional (3D) representation of the surgical scene. In some embodiments, the one or more two-dimensional images may not comprise a superimposed image.

In some embodiments, the image processing module may be configured to simulate a plurality of virtual light sources configured to generate one or more virtual light beams that intersect the one or more two-dimensional (2D) images of the surgical scene. In some embodiments, the one or more virtual light beams may be parallel.

In some embodiments, the image processing module may be configured to generate the one or more virtual shadows using a modified image of the surgical scene, which modified image may be derived by rotating the one or more two-dimensional (2D) images to align two or more pixels of the 2D images with the one or more virtual light beams.

In some embodiments, the image processing module may be configured to implement a shadow mapping algorithm to generate the one or more virtual shadows.

In some embodiments, the image processing module may be configured to use the modified image to (i) improve an efficiency of the shadow mapping algorithm and (ii) reduce an amount of computation required to generate the one or more virtual shadows.

In some embodiments, the plurality of virtual light sources may be repositionable to generate the one or more virtual shadows in different regions of interest within the one or more two-dimensional (2D) images of the surgical scene.

In some embodiments, the scope may comprise a laparoscope, an endoscope, a borescope, a videoscope, or a fiberscope.

In some embodiments, the depth information and the topology of the surgical scene may be obtained using a time of flight (TOF) sensor. In some embodiments, the TOF sensor may be integrated with the imaging device. In some embodiments, the imaging device may be integrated with the scope.

In some embodiments, the one or more two-dimensional (2D) images may be rotated by a rotational angle that is computed based on an angle formed between (i) a reference line comprising two or more pixels within the 2D images and (ii) a projection of the one or more virtual light beams onto a reference plane containing the 2D images.

In another aspect, the present disclosure provides a method for enhancing medical images. The method may comprise: obtaining an initial image of a surgical scene and a depth map associated with the initial image; and adjusting a shading of one or more pixels of the initial image based at least in part on (i) a light intensity fall-off pattern and (ii) the depth map associated with the initial image of the surgical scene, to generate an updated image having adjusted shading.

In some embodiments, the updated image having the adjusted shading may provide enhanced depth perception to aid a surgical procedure at or near the surgical scene. In some embodiments, adjusting the shading of the one or more pixels may involve (i) computing surface normal information from the depth map and (ii) using the surface normal information to remove existing shading in the initial image due to a topology of the surgical scene, thereby creating an unshaded image.

In some embodiments, a shading based on the surface normal information may be applied to the unshaded image to generate the updated image having the adjusted shading.

In some embodiments, the initial image of the surgical scene may be obtained by illuminating the surgical scene with light directed from an illumination source via a scope.

In some embodiments, adjusting the shading of the one or more pixels may involve calibrating an illumination of the initial image at a plurality of depths to remove one or more illumination effects generated by the illumination source within the initial image, thereby creating an unshaded image.

In some embodiments, a shading based on surface normal information derived from the depth map may be applied to the unshaded image to generate the updated image having the adjusted shading.

In some embodiments, the light intensity fall-off pattern may be a function of (i) a vertical distance from a tip of the scope to a center point of illumination within the surgical scene and (ii) a horizontal distance from the center point of illumination to the one or more pixels of the initial image.

In some embodiments, adjusting the shading of the one or more pixels based on the depth map may comprise modifying the shading of the one or more pixels based at least in part on a relative distance or a relative orientation of one or more features associated with the one or more pixels, in relation to a tip of the scope.

In some embodiments, adjusting the shading of the one or more pixels may comprise modifying a color intensity, a brightness, or an opacity of the one or more pixels.

In some embodiments, the initial image and the updated image may comprise at least a portion of a laser speckle contrast image.

In some embodiments, the initial image and the updated image may comprise a physiological visualization of a perfusion pattern obtained from a laser speckle contrast image or a tissue classification obtained from hyperspectral imaging.

In some embodiments, the scope may comprise a laparoscope, an endoscope, a borescope, a videoscope, or a fiberscope.

In some embodiments, the depth map may be obtained using a time of flight (TOF) sensor or a stereoscopic camera.

In some embodiments, the method may further comprise using surface normal information obtained from the depth map to generate an unshaded image of the surgical scene from the initial image. In some embodiments, the unshaded image may comprise an image of the surgical scene with a uniform color intensity.

In some embodiments, the method may further comprise simulating one or more virtual light sources and modifying a shading of one or more pixels of the unshaded image based on a relative position or a relative orientation of the one or more virtual light sources in relation to the one or more pixels within the unshaded image.

In some embodiments, the one or more virtual light sources may be repositionable relative to the unshaded image of the surgical scene.

In some embodiments, the method may further comprise using the initial image and the updated image to compute a blood flow velocity through one or more blood vessels within the surgical scene, based in part on (i) a size of the one or more blood vessels and (ii) a concentration of blood within the one or more blood vessels.

In another aspect, the present disclosure provides a system for enhancing medical images. The system may comprise: an image processing module comprising one or more processors that, upon execution of a set of instructions stored in memory, are configured to: (i) obtain an initial image of a surgical scene and a depth map associated with the initial image; and (ii) adjust a shading of one or more pixels of the initial image based on (i) a light intensity fall-off pattern and (ii) the depth map, to generate an updated image having adjusted shading.

In some embodiments, adjusting the shading of the one or more pixels may comprise using surface normal information obtained from the depth map to generate an unshaded image of the surgical scene from the initial image, wherein the unshaded image comprises an image of the surgical scene with a uniform color intensity.

In another aspect, the present disclosure provides a method for augmented medical imaging. The method may comprise: (a) obtaining an initial scope image of a deformable tissue surface, an initial depth map corresponding to the initial scope image, and an initial pre-operative image of the deformable tissue surface; (b) overlaying the initial pre-operative image onto the initial scope image, or vice versa, to generate an initial superimposed image; (c) obtaining a subsequent scope image of the deformable tissue surface and an updated depth map corresponding to the subsequent scope image; (d) computing a deformation delta based at least in part on the initial depth map and the updated depth map; (e) using the deformation delta to generate a modified pre-operative image from the initial pre-operative image; and (f) overlaying the modified pre-operative image onto the subsequent scope image, or vice versa, to generate an updated superimposed image that corresponds to the deformable tissue surface in a deformed state.

In some embodiments, the steps of (c) - (f) may be performed substantially in real-time for a series of subsequent scope images. In some embodiments, the series of subsequent scope images may comprise one or more scope images taken while the deformable tissue surface undergoes a deformation.

In some embodiments, the initial scope image may correspond to an image of the deformable tissue surface in an undeformed state. In some embodiments, the subsequent scope image may correspond to an image of the deformable tissue surface in a deformed state. In some embodiments, the initial pre-operative image may comprise a pre-operative scan selected from the group consisting of a computed tomographic (CT) scan, a magnetic resonance imaging (MRI) scan, and an ultrasonography scan.

In another aspect, the present disclosure provides a method for augmented medical imaging. The method may comprise: (a) obtaining an initial scope image of a deformable tissue surface, an initial depth map of the deformable tissue surface, and an initial pre-operative image of the deformable tissue surface; (b) overlaying the initial pre-operative image onto the initial scope image, or vice versa, to generate an initial superimposed image; (c) obtaining a subsequent scope image of the deformable tissue surface and an updated depth map of the deformable tissue surface; (d) computing a deformation delta based at least in part on the initial depth map and the updated depth map; and (e) using the deformation delta to generate an updated superimposed image from the initial superimposed image, wherein the updated superimposed image corresponds to the deformable tissue surface in a deformed state.

In some embodiments, the steps of (c) - (e) may be performed substantially in real-time for a series of subsequent scope images. In some embodiments, the series of subsequent scope images may comprise one or more scope images taken while the deformable tissue surface undergoes a deformation. In some embodiments, the initial scope image may correspond to an image of the deformable tissue surface in an undeformed state. In some embodiments, the subsequent scope image may correspond to an image of the deformable tissue surface in a deformed state. In some embodiments, the initial pre-operative image may comprise a pre-operative scan selected from the group consisting of a computed tomographic (CT) scan, a magnetic resonance imaging (MRI) scan, and an ultrasonography scan.

In another aspect, the present disclosure provides a method for augmented medical imaging. The method may comprise: (a) obtaining an initial scope image of a deformable tissue surface and an initial pre-operative image of the deformable tissue surface; (b) generating an initial depth map of the deformable tissue surface based at least in part on the initial scope image; (c) identifying a first set of target points on the initial scope image and the initial pre-operative image, wherein the first set of target points comprises at least one similar feature in both the initial scope image and the initial pre-operative image; (d) using the first set of target points to register and overlay the initial pre-operative image onto the initial scope image; (e) obtaining a subsequent scope image of the deformable tissue surface; (f) generating an updated depth map of the deformable tissue surface based at least in part on the subsequent scope image; (g) computing a deformation delta based at least in part on the initial depth map and the updated depth map; (h) using the deformation delta to generate a modified pre-operative image from the initial pre-operative image and identify a second set of target points in the subsequent scope image and the modified pre-operative image, wherein the second set of target points comprises the at least one similar feature associated with the first set of target points; and (i) using the second set of target points to register and overlay the modified pre-operative image onto the subsequent scope image.

In some embodiments, the steps of (e) - (i) may be performed substantially in real-time for a series of subsequent scope images. In some embodiments, the series of subsequent scope images may comprise one or more scope images taken while the deformable tissue surface undergoes a deformation.

In some embodiments, the first set of target points and the second set of target points may correspond to at least a portion of a blood perfusion pattern.

In some embodiments, the initial scope image and the subsequent scope image may be obtained using an imaging device and a scope. In some embodiments, the scope may be selected from the group consisting of a laparoscope, an endoscope, a borescope, a videoscope, and a fiberscope. In some embodiments, the imaging device may be integrated with the scope.

In some embodiments, the initial depth map and the updated depth map may be obtained using a time of flight (TOF) sensor. In some embodiments, the TOF sensor may be integrated with the imaging device. In some embodiments, the initial pre-operative image may comprise a pre-operative scan selected from the group consisting of a computed tomographic (CT) scan, a magnetic resonance imaging (MRI) scan, and an ultrasonography scan.

In some embodiments, the initial scope image may correspond to an image of the deformable tissue surface in an undeformed state. In some embodiments, the subsequent scope image may correspond to an image of the deformable tissue surface in a deformed state.

In another aspect, the present disclosure provides a method for augmented medical imaging. The method may comprise: (a) obtaining an initial scope image of a deformable tissue surface and an initial pre-operative image of the deformable tissue surface; (b) generating an initial depth map of the deformable tissue surface from the initial scope image; (c) identifying one or more points of interest on the initial scope image and the initial pre-operative image, wherein the one or more points of interest comprise at least one similar feature in both the initial scope image and the initial pre-operative image; (d) using the one or more points of interest to register and overlay the initial pre-operative image onto the initial scope image, thereby generating an overlaid image; (e) obtaining a subsequent scope image of the deformable tissue surface; (f) generating an updated depth map of the deformable tissue surface using at least the subsequent scope image; (g) computing a deformation delta based at least in part on the initial depth map and the updated depth map; and (h) using the deformation delta to generate an updated overlaid image based at least in part on the overlaid image, wherein the updated overlaid image corresponds to the deformable tissue surface in a deformed state.

In some embodiments, the initial scope image and the subsequent scope image may be obtained using a scope selected from the group consisting of a laparoscope, an endoscope, a borescope, a videoscope, and a fiberscope.

In another aspect, the present disclosure provides a system for augmented medical imaging. The system may comprise: (a) an imaging device configured to obtain an initial scope image of a deformable tissue surface and a subsequent scope image of the deformable tissue surface; (b) a depth sensor configured to generate an initial depth map of the deformable tissue surface using the initial scope image and an updated depth map of the deformable tissue surface using the subsequent scope image; and (c) an image processing module configured to: overlay an initial pre-operative image of the deformable tissue surface onto the initial scope image, or vice versa, based at least in part on a first set of target points identified in the initial scope image and the initial pre-operative image, wherein the first set of target points comprises at least one similar feature in both the initial scope image and the initial pre-operative image; compute a deformation delta based at least in part on the initial depth map and the updated depth map; and use the deformation delta to (i) generate a modified pre-operative image from the initial pre-operative image and (ii) overlay the modified pre-operative image onto the subsequent scope image based at least in part on a second set of target points in the subsequent scope image and the modified pre-operative image, wherein the second set of target points comprises the at least one similar feature associated with the first set of target points.

In some embodiments, the imaging device may be configured to obtain the initial scope image and the subsequent scope image via a scope. In some embodiments, the scope may be selected from the group consisting of a laparoscope, an endoscope, a borescope, a videoscope, and a fiberscope. In some embodiments, the imaging device may be integrated with the scope.

In some embodiments, the depth sensor may comprise a time of flight (TOF) sensor.

In some embodiments, the initial pre-operative image may comprise a pre-operative scan selected from the group consisting of a computed tomographic (CT) scan, a magnetic resonance imaging (MRI) scan, and an ultrasonography scan.

In some embodiments, the initial scope image may comprise an image of the deformable tissue surface in an undeformed state. In some embodiments, the subsequent scope image may comprise an image of the deformable tissue surface in a deformed state.

In another aspect, the present disclosure provides a method for augmented medical imaging. The method may comprise: (a) computing a deformation delta based at least in part on an initial depth map of a deformable tissue surface and an updated depth map of the deformable tissue surface; and (b) using the deformation delta to generate an updated superimposed image from an initial superimposed image.

In some embodiments, the initial superimposed image may be generated by overlaying an initial pre-operative image of the deformable tissue surface onto an initial scope image of the deformable tissue surface, or by overlaying the initial scope image of the deformable tissue surface onto the initial pre-operative image of the deformable tissue surface.

In some embodiments, the initial depth map of the deformable tissue surface may be generated using at least the initial scope image. In some embodiments, the updated depth map of the deformable tissue surface may be generated using at least one subsequent scope image that is captured after the initial scope image.

In some embodiments, the initial superimposed image may correspond to the deformable tissue surface in an undeformed state. In some embodiments, the updated superimposed image may correspond to the deformable tissue surface in a deformed state.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in the art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 schematically illustrates an imaging device and a scope, in accordance with some embodiments.

FIG. 2 schematically illustrates a plurality of virtual light sources, in accordance with some embodiments.

FIG. 3 schematically illustrates a reference projection line and a reference pixel line, in accordance with some embodiments.

FIG. 4 schematically illustrates a rotated image and a rotated depth map, in accordance with some embodiments.

FIG. 5 schematically illustrates a virtual shadow in a region of interest, in accordance with some embodiments.

FIG. 6 schematically illustrates a plurality of virtual light sources that are repositioned relative to the image and the depth map, in accordance with some embodiments.

FIG. 7 schematically illustrates an exemplary method for enhancing depth perception, in accordance with some embodiments.

FIG. 8 schematically illustrates a computer system that is programmed or otherwise configured to implement methods provided herein, in accordance with some embodiments.

FIG. 9 schematically illustrates an illumination fall-off pattern within an image of a surgical scene, in accordance with some embodiments.

FIG. 10A schematically illustrates an example of an initial image of a surgical scene with a radial shading gradient, in accordance with some embodiments.

FIG. 10B schematically illustrates an unshaded image, in accordance with some embodiments.

FIG. 10C schematically illustrates an updated image with adjusted shading, in accordance with some embodiments.

FIG. 11A schematically illustrates a deformable tissue surface in an undeformed state, in accordance with some embodiments.

FIG. 11B schematically illustrates a deformable tissue surface in a deformed state, in accordance with some embodiments.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the embodiments of the present disclosure. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure provides systems and methods for improving medical imaging technology. The systems and methods disclosed herein may be used to enhance medical imaging by selectively generating virtual shadows within medical images to provide an operator with enhanced depth perception. Further, the system and methods disclosed herein may be implemented to selectively position and reposition one or more virtual shadows, thereby allowing medical operators to visualize anatomical structures without having one or more virtual shadows occlude a region of interest and without losing monocular cues that can aid in depth perception. The systems and methods disclosed herein may also be implemented to adjust the shading in medical images to augment depth perception. In some cases, the systems and methods disclosed herein may be used for dynamic, real-time augmented reality image overlays for deformable tissue regions undergoing physical deformations. As such, the systems and methods disclosed herein can provide medical operators with additional visual information that can enhance depth perception and inform or guide them during a surgical procedure.

In an aspect, the present disclosure provides a method for enhancing depth perception to aid a surgical procedure. The method may comprise (a) using a scope and an imaging device to obtain (i) an image of a surgical scene and (ii) a depth map associated with the image.

The scope may comprise a laparoscope, an endoscope, a borescope, a videoscope, or a fiberscope. The scope may be optically coupled to an imaging device. When optically coupled with the scope, the imaging device may be configured to obtain one or more images through a hollow inner region of the scope. The imaging device may comprise a camera, a video camera, a three-dimensional (3D) depth camera, a stereo camera, a depth camera, a Red Green Blue Depth (RGB-D) camera, a time-of-flight (TOF) camera, an infrared camera, a charge coupled device (CCD) image sensor, or a complementary metal oxide semiconductor (CMOS) image sensor.

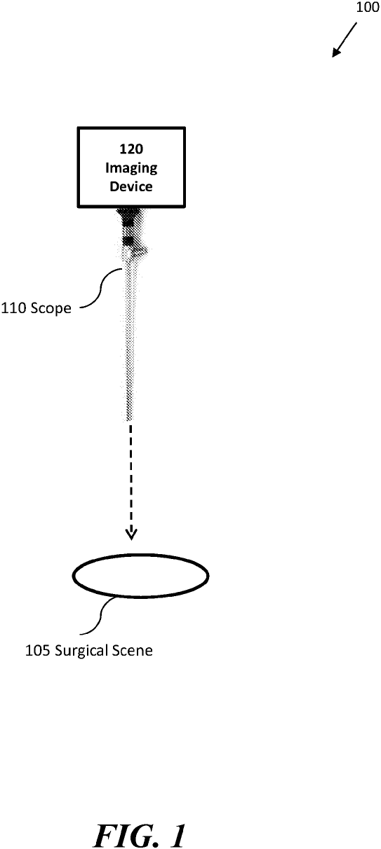

FIG. 1 illustrates a system 100 for enhancing depth perception to aid in a surgical procedure. The surgical procedure may comprise one or more medical operations performed on a surgical site or a surgical scene 105 of a patient. The system 100 may comprise a scope 110 and an imaging device 120 optically coupled to the scope 110. In some cases, the imaging device 120 may be integrated with the scope 110. The imaging device 120 may be configured to obtain one or more images of a surgical scene 105 of a patient. The surgical scene 105 may comprise a portion of an organ of a patient or an anatomical feature or structure within a patient’s body. The surgical scene 105 may comprise a surface of a tissue of the patient’s body. The surface of the tissue may comprise epithelial tissue, connective tissue, muscle tissue (e.g., skeletal muscle tissue, smooth muscle tissue, and/or cardiac muscle tissue), and/or nerve tissue. In some cases, the surgical scene 105 may comprise one or more critical structures, such as cancer tissue, arteries, veins, nerves, a ureter, and/or a bile duct. In some cases, the surgical scene 105 may comprise one or more perfusion patterns showing a flow of a bodily fluid within a subject. The bodily fluid may comprise, for example, blood, urine, lymph, tissue fluid, milk, saliva, semen, and/or bile. In any of the embodiments described herein, the surgical scene 105 may comprise one or more physiologic visualizations, pathologic visualizations, morphologic visualizations, and/or anatomic visualizations.

In some cases, the surgical scene may be a region within a subject (e.g., a human, a child, an adult, a medical patient, a surgical patient, etc.) that may be illuminated by one or more illumination sources. The surgical scene may be a region within the subject’s body. In some cases, the surgical scene may correspond to an organ of the subject, a vasculature of the subject, or any anatomical feature or structure of the subject’s body. In some cases, the surgical scene may correspond to a portion of an organ, a vasculature, or an anatomical structure of the subject. In some cases, the surgical scene may comprise one or more internal bodily processes or phenomena associated with a physiological and/or a pathological characteristic or condition of a subject.

In some cases, the surgical scene may be a tissue region of or within the subject’s body. The region may comprise a portion of an epidermis, a dermis, and/or a hypodermis of the subject. In other cases, the surgical scene may correspond to a wound located on the subject’s body. The wound may be a burn wound. Alternatively, the surgical scene may correspond to an amputation site of the subject. In any of the embodiments described herein, the surgical scene may correspond to a portion of a subject’s body that receives blood flow. In any of the embodiments described herein, the surgical scene may correspond to a region of or within a subject’s body through which a biological material or fluid may be configured to move or flow.

The imaging device may be configured to obtain one or more images of a surgical scene on or in a patient’s body. The one or more images may comprise a two-dimensional (2D) image or a three-dimensional (3D) image of a surgical scene on or in the patient’s body. The one or more images of the surgical scene may be processed to generate one or more virtual shadows within one or more portions of the images of the surgical scene. The one or more images of the surgical scene may not or need not comprise a pre-operative scan. A pre-operative scan may comprise a computed tomographic (CT) scan, a magnetic resonance imaging (MRI) scan, or an ultrasonography scan. In some alternative embodiments, the one or more images of the surgical scene may be overlaid onto a pre-operative scan of the surgical scene, or vice versa, to generate a superimposed or overlaid image. A superimposed image may comprise an overlay of (i) the image of the surgical scene on (ii) a pre-operative scan, or vice versa. In some cases, the system and methods disclosed herein may be used to generate one or more virtual shadows within the superimposed image.

The imaging device may be configured to obtain one or more depth maps of the surgical scene. The one or more depth maps may be associated with the one or more images of the surgical scene. The one or more depth maps may comprise an image or an image channel that contains information relating to a distance or a depth of one or more surfaces within the surgical scene from a reference viewpoint. The reference viewpoint may correspond to a location of the imaging device relative to one or more portions of the surgical scene. The one or more depth maps may comprise depth values for a plurality of points or locations within the surgical scene. The one or more depth maps may comprise depth values for a plurality of pixels within the image of the surgical scene. The depth values may correspond to a distance from the imaging device to a plurality of points or locations within the surgical scene. The depth values may correspond to a distance from a virtual viewpoint to a plurality of pixels within an image of the surgical scene. In some cases, the virtual viewpoint may correspond to a position and/or an orientation of the imaging device in real space.

In some cases, the depth map may be obtained using a time of flight (TOF) sensor. The TOF sensor may be integrated with the imaging device. The TOF sensor may be configured to obtain and/or generate a depth map based in part on a time it takes for light (e.g., a light wave, a light pulse, or a light beam) to travel from one or more portions of a surface of the surgical scene to a detector of the TOF sensor after being reflected off of the one or more portions of the surface of the surgical scene. In some cases, the depth map may be obtained using a stereoscopic camera.

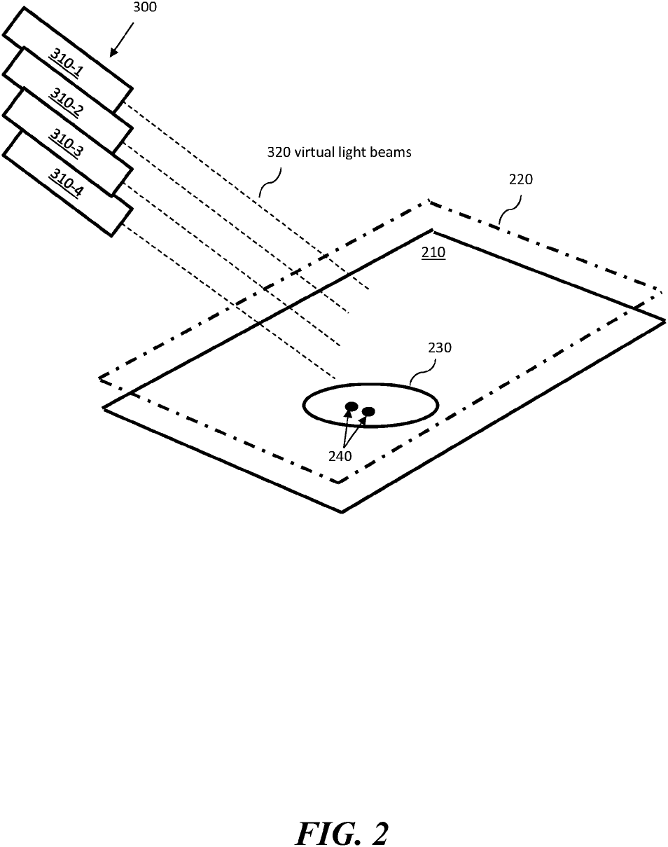

In some embodiments, the method may further comprise (b) identifying a region of interest within the image or the depth map. The region of interest may comprise a plurality of pixels. FIG. 2 illustrates an image 210 of a surgical scene and a depth map 220 associated with the image 210 of the surgical scene. In some cases, a user or an operator of the systems may select and/or identify a region of interest 230 within the image 210 or the depth map 220 of the surgical scene. The region of interest 230 may comprise a plurality of pixels 240. The plurality of pixels 240 may be arranged in a rectilinear array. The plurality of pixels 240 may correspond to one or more portions of a surface of a tissue of the patient. The plurality of pixels 240 may correspond to one or more locations on a portion of an organ of the patient. The plurality of pixels 240 may correspond to one or more locations on a portion of an anatomical feature or structure of the patient’s body.

In some embodiments, the method may further comprise (c) simulating a virtual light model. The virtual light model may be simulated using an image processing algorithm. The virtual light model may be a computer-generated representation of light (e.g., a point light source, a sun light source, a spotlight light source, and/or an area light source) that is configured to simulate one or more lighting or shading effects in a computer-generated three-dimensional (3D) scene. The virtual light model may comprise a plurality of virtual light sources configured to generate one or more virtual light beams that intersect the image of the surgical scene. The virtual light model may be simulated within a computer-generated 3D scene. The computer-generated 3D scene may be a computer-generated virtual 3D space. The computer-generated virtual 3D space may be a virtual representation of a real space in which a medical operator is operating on a surgical scene. The virtual 3D space may be scaled to provide a similar and/or proportional representation of the surgical scene and/or any tools that may be present in the vicinity of the surgical scene. The computer-generated virtual 3D space may be an imaginary or virtual space that can accommodate a placement of virtual objects (e.g., a virtual light model) in a location within the computer-generated virtual 3D space. The location within the computer-generated virtual 3D space may be defined using a three-dimensional cartesian coordinate system (i.e., X, Y, and Z coordinates), a cylindrical coordinate system, and/or a spherical coordinate system. An image or a depth map of a surgical scene of a patient may be provided within the virtual 3D space for image processing. The virtual light model may be configured to interact with the image or the depth map to produce one or more lighting or shading effects within a portion of the image or the depth map of the surgical scene.

FIG. 2 illustrates a virtual light model 300 comprising a plurality of virtual light sources 310-1, 310-2, 310-3, and 310-4. The plurality of virtual light sources may comprise one or more virtual light sources. In some cases, the plurality of virtual light sources may comprise one virtual light source, two virtual light sources, three virtual light sources, four virtual light sources, five virtual light sources, six virtual light sources, seven virtual light sources, eight virtual light sources, nine virtual light sources, ten virtual light sources, or more. As described herein, a virtual light source may be a virtual (i.e., computer-generated) representation of light originating from a location within or near a computer-generated three-dimensional (3D) scene. The virtual light source may comprise a point light source, a sun light source, a spotlight light source, and/or an area light source that is configured to provide a lighting or shading effect within the computer-generated 3D scene. A point light source may be modeled as a light that is positioned within the computer-generated 3D scene at a specific location and shines light equally in all directions. A sun light source may be modeled as light that is positioned outside the 3D scene and far enough away that all rays of light propagate along a same direction. A spotlight light source may be modeled as a light that is focused and forms a cone-shaped envelope as it projects out from the spotlight light source. An area light source may be modeled as light that originates from a rectangular area and projects light from one side of the rectangular area.

In some embodiments, the plurality of virtual light sources may be arranged in a lateral or side-by-side configuration. In other embodiments, the plurality of virtual light sources may be arranged in a ring configuration such that each of the plurality of virtual light sources is equidistant from a center point. Alternatively, the plurality of virtual light sources may be arranged in a pre-determined pattern. The pre-determined pattern may correspond to a shape of a circle, a triangle, a square, a rectangle, or any polygon having three or more sides. The plurality of virtual light sources may be arranged at one or more distances and/or one or more orientations relative to a reference point on the image or the depth map. The one or more distances and the one or more orientations may be the same. Alternatively, the one or more distances and the one or more orientations may be different. The reference point may correspond to a portion of the image or the depth map. In some cases, the reference point may correspond to one or more pixels of the image or the depth map. The plurality of virtual light sources may be repositionable at any distance and/or any orientation relative to the reference point.

As described above, each of the plurality of virtual light sources may be arranged in a side-by-side or lateral configuration. In some cases, each of the plurality of virtual light sources may be separated by a same separation distance. In other cases, each of the plurality of virtual light sources may be separated by one or more distinct separation distances.

In any of the embodiments described herein, the plurality of virtual light sources may be arranged such that each of the plurality of virtual light sources is disposed at the same distance from a reference point on the image or the depth map. In some cases, the plurality of virtual light sources may be arranged such that each of the plurality of virtual light sources is disposed at one or more distinct distances from the reference point.

As shown in FIG. 2, the plurality of virtual light sources 310-1, 310-2, 310-3, and 310-4 may be configured to generate one or more virtual light beams 320. A virtual light beam may be a vector within the computer-generated three-dimensional (3D) scene that represents a ray of light originating from the one or more virtual light sources. The virtual light beam may illuminate a portion of an image or a depth map that coincides with the virtual light beam. The image or the depth map of the surgical scene may be provided within the same virtual 3D space containing the plurality of virtual light sources. The virtual light beam may produce one or more shading or lighting effects within a portion of an image or a depth map (e.g., a pixel or a group of pixels within the image or the depth map) that the virtual light beam intersects. The one or more virtual light beams 320 may be parallel to one another. Alternatively, the one or more virtual light beams 320 may not or need not be parallel to one another. The one or more virtual light beams 320 may intersect the image 210 of the surgical scene or the depth map 220 associated with the image 210 of the surgical scene. The image or the depth map of the surgical scene may be provided on a reference plane within the virtual 3D space for image processing. The one or more virtual light beams 320 may intersect the reference plane containing the image 210 or the depth map 220 of the surgical scene at an angle of incidence relative to the reference plane. The angle of incidence may be greater than 0 degrees and less than 180 degrees. The angle of incidence may be at least about 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, or more. In some preferred embodiments, the angle of incidence may range from about 5 degrees to about 85 degrees.

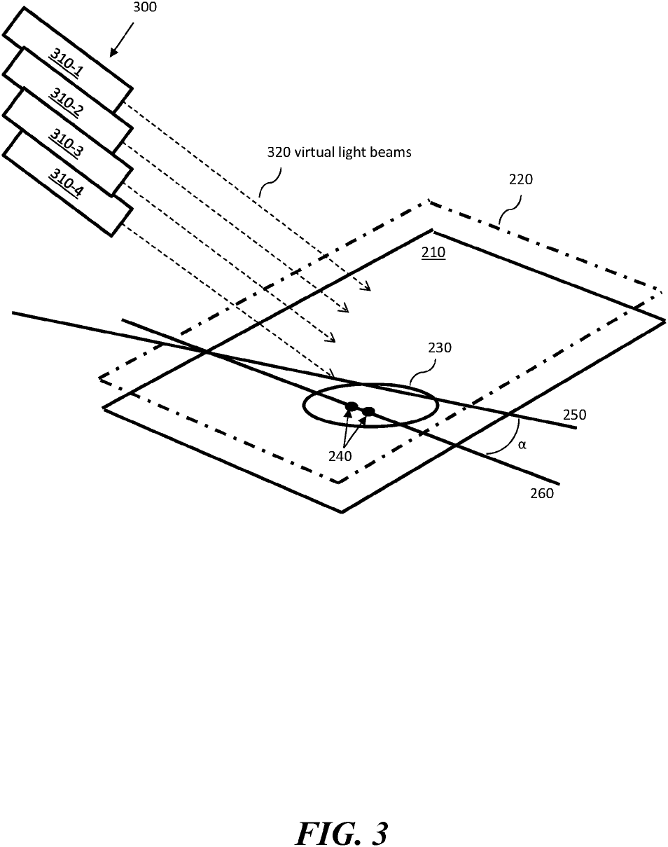

As illustrated in FIG. 3, one or more virtual light beams 320 may be directed towards the image 210 or the depth map 220. The one or more virtual light beams may extend and/or propagate along a direction of travel within the computer-generated three-dimensional (3D) scene. The direction of travel may be represented by one or more vectors in virtual 3D space. The one or more vectors may correspond to a direction of travel of the one or more virtual light beams in the virtual three-dimensional space. The one or more vectors may intersect the image 210 or the depth map 220 at an angle of incidence, as described above. The one or more vectors may be projected onto an XY-plane corresponding to a plane of the image 210 or the depth map 220. The projection of the one or more vectors onto the XY-plane may produce a reference projection line 250 that is located on the XY-plane. The reference projection line 250 may form an offset angle α relative to a pixel reference line 260. The pixel reference line 260 may correspond to a line formed between two or more pixels 240 in the image 210 or the depth map 220. The pixel reference line 260 may correspond to a line formed between two or more pixels 240 in a region of interest 230 within the image 210 or the depth map 220. The offset angle may be greater than 0 degrees and less than 180 degrees. The offset angle may be at least about 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, or more. In some preferred embodiments, the offset angle may range from about 5 degrees to about 175 degrees.

In some embodiments, the method may further comprise (d) rotating the depth map and/or the image of the surgical scene by a rotational angle to align the plurality of pixels with the one or more virtual light beams. The depth map and/or the image of the surgical scene may be rotated using an image processing algorithm. The rotational angle may be computed based in part on an angle formed between (i) a reference line comprising two or more pixels within the region of interest and (ii) a projection of the one or more virtual light beams onto a reference plane containing the image of the surgical scene. The rotational angle may correspond to the offset angle α shown in FIG. 3 and described above. The reference line comprising two or more pixels within the region of interest may correspond to the pixel reference line 260 shown in FIG. 3 and described above. The projection of the one or more virtual light beams onto a reference plane containing the image of the surgical scene may correspond to the reference projection line 250 shown in FIG. 3 and described above. Aligning the plurality of pixels with the one or more virtual light beams may involve rotating the image or the depth map about the Z-axis such that two or more pixels within a region of interest are aligned with one or more reference projection lines located on a reference plane containing the image or the depth map. The two or more pixels may be arranged with a row of pixels or a column of pixels. As described above, the one or more reference projection lines may be produced when one or more vectors corresponding to a direction of travel of the one or more virtual light beams are projected onto a reference XY-plane containing the image or the depth map. The projection of the one or more vectors or rays onto the reference XY-plane may produce one or more reference projection lines located on the reference XY-plane containing the image and/or the depth map.

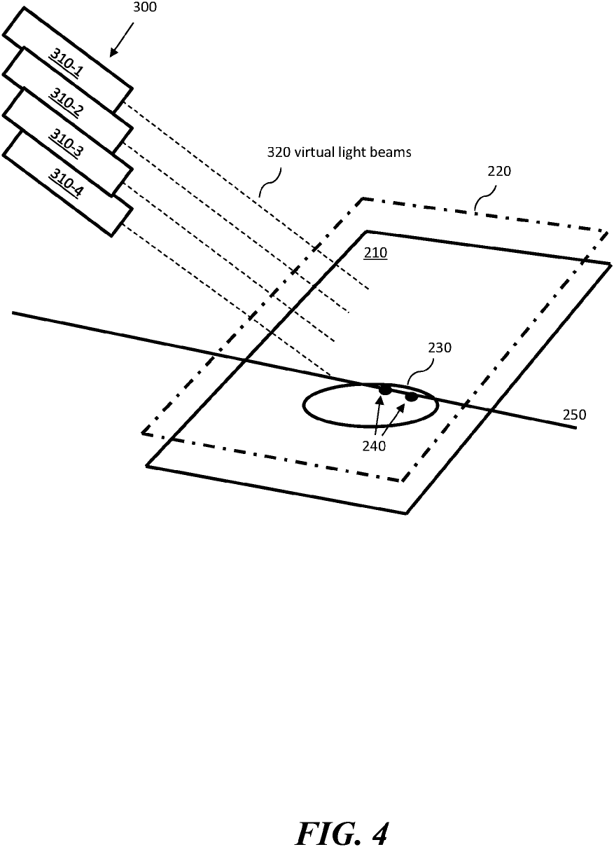

FIG. 4 illustrates an image 210 and a depth map 220 rotated by an offset angle α. After a rotation of the image 210 and the depth map 220, two or more pixels 240 within the region of interest 230 may be aligned with and/or may coincide with the reference projection line 250.

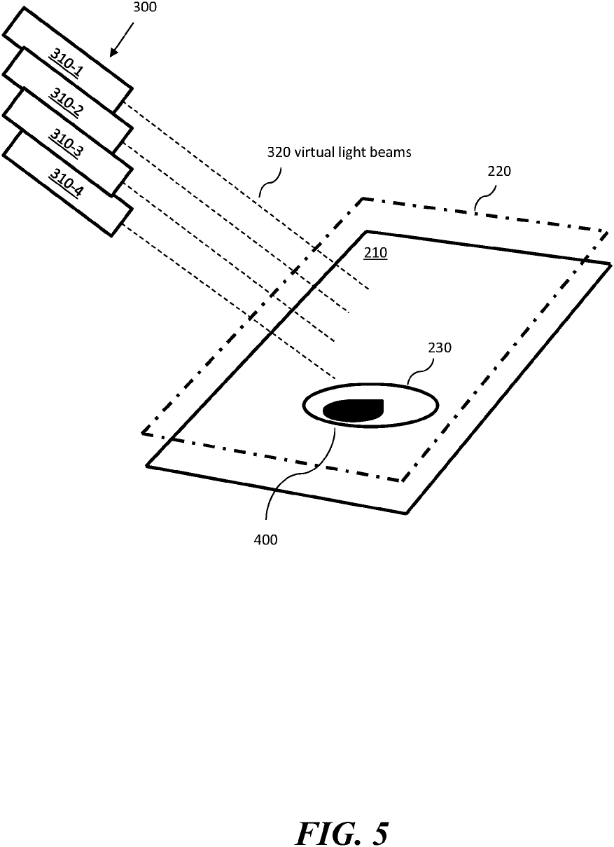

In some embodiments, the method may further comprise (e) using an image processing algorithm to generate one or more virtual shadows for one or more portions of the region of interest, based in part on the rotated image and the rotated depth map. A virtual shadow may be a shaded or darkened portion within the rotated image and/or the rotated depth map that is generated based on an interaction of one or more virtual light beams with a portion of the rotated image or the rotated depth map. FIG. 5 illustrates a virtual shadow 400 that may be generated within a region of interest 230 of the rotated image 210 or the rotated depth map 220 using the shadow mapping algorithm. Generating the one or more virtual shadows may involve adjusting a brightness, a color, an opacity, and/or a shading of one or more pixels in the rotated image of the surgical scene.

The one or more virtual shadows may be generated when a portion of the surgical scene is blocked or partially blocked from the one or more virtual light beams by another portion of the surgical scene. The virtual shadows may be generated in part based on a topography of the surgical scene and/or a geometry of a portion of the surgical scene. The topography of the surgical scene and/or the geometry of the portion of the surgical scene may be derived from the image or the depth map of the surgical scene. The one or more virtual shadows may be generated to enhance depth perception within the image of the surgical scene and to aid a surgical procedure in or near the surgical scene. In some cases, the one or more virtual shadows may be used to indicate whether a tool used by a medical operator is in contact with one or more portions of the surgical scene.

The image processing algorithm may be configured to implement a shadow mapping algorithm to generate the one or more virtual shadows. The shadow mapping algorithm may be configured to generate a shadow map for the image of the surgical scene and/or a rotated image of the surgical scene. The shadow map may be used to draw the one or more virtual shadows within a portion of the image or the rotated image. The shadow map may comprise one or more shadow map values for each pixel within the image and/or the rotated image. The one or more shadow map values may comprise one or more numerical values indicating a color, an opacity, a level of brightness, and/or a degree of shading of one or more pixels within the image and/or the rotated image. The one or more shadow map values may be computed for one or more pixels within a portion of the image and/or the rotated image that corresponds to a virtual shadow. In some cases, the one or more shadow map values may be computed for one or more pixels within a portion of the image and/or the rotated image that does not correspond to a virtual shadow.

The shadow map may be generated based in part on one or more shadow masks computed by the image processing algorithm. The one or more shadow masks may indicate the presence of a shadow or a lack of a presence of a shadow for one or more pixels within an image of a surgical scene or a portion thereof. In some cases, one or more shadow masks may be computed for each simulated virtual light source that produces a shading or lighting effect. The one or more shadow masks may be computed using path tracing. Path tracing may involve defining, for each pixel in the image, a path vector that points to the simulated virtual light source. Path tracing may further involve determining if any other pixels fall on a path vector associated with a certain pixel. Every pixel whose path vector is obstructed by another pixel may be categorized as a shadow pixel, and a corresponding shadow mask may indicate that the pixel should have a shadow drawn on the pixel. In some cases, the shadow map may be generated by processing one or more shadow masks. In other cases, the shadow map may be generated by aggregating, combining, comparing, and/or processing two or more shadow masks.

In some embodiments, the shadow mapping algorithm may be configured to compute shadow maps and generate virtual shadows based in part on (i) a position and an orientation of the one or more virtual light sources relative to a region of interest within the rotated image and/or the rotated depth map and/or (ii) a comparison of depth map values or shadow map values for two or more pixels within the region of interest. The two or more pixels may comprise two or more adjacent pixels within a row of pixels or a column of pixels. In some cases, a virtual shadow may be drawn for a first pixel when the first pixel has a greater depth map value than a second pixel that is positioned between the first pixel and the virtual light source.

In some embodiments, the image processing algorithm may be configured to generate the one or more virtual shadows based in part on an eroded depth map. The image processing algorithm may be configured to generate an eroded depth map using the rotated image and/or the rotated depth map. The eroded depth map may comprise a depth map having updated depth map values and/or updated shadow map values for one or more pixels within the rotated image and/or the rotated depth map. The updated depth map values and/or the updated shadow map values may be computed in part based on a comparison of depth map values for the one or more pixels against shadow map values of one or more neighboring pixels adjacent to the one or more pixels. The eroded depth map may be generated in part based on a comparison of depth map values and shadow map values for a plurality of pixels located along a shadow slope extending from a virtual light source towards the plurality of pixels within the rotated image and/or the rotated depth map. The eroded depth map may be generated by comparing a depth value of a first pixel against a shadow map value of a second pixel that is positioned in front of the first pixel. The second pixel may lie along the shadow slope and may be positioned between the first pixel and the virtual light source. The depth map value of the first pixel may be replaced by the greater of the depth map value of the first pixel and the shadow map value of a previous pixel (i.e., the second pixel). The image processing algorithm may be configured to compare the depth map to the eroded depth map. Based on a comparison of the depth map to the eroded depth map, the image processing algorithm may be configured to draw a shadow for each pixel with a depth map value that is greater than the corresponding eroded depth map value. In some embodiments, the image processing algorithm may be configured to revert a rotation of the image after computing or generating one or more virtual shadows within a portion of the rotated image.

The image processing algorithm may be configured to optimize a computation or generation of the one or more virtual shadow by rotating the image of the surgical scene and/or by rotating the depth map before computing or generating the one or more virtual shadows. The rotated image and/or the rotated depth map may be derived by rotating the image or the depth map such that a dimension (i.e., a height or a width) of the image or the depth map is (i) parallel to an orientation of the virtual light source or (ii) parallel to a direction of travel of one or more virtual light beams generated by the virtual light source. When rotated, a top row of pixels of the rotated image and/or the rotated depth map may be positioned closest to the virtual light source. When rotated, one or more columns of pixels within the rotated image and/or the rotated depth map may be (i) parallel to an orientation of the virtual light source or (ii) parallel to a direction of travel of one or more virtual light beams generated by the virtual light source. The rotated image and/or the rotated depth map may be derived by rotating the image or the depth map by an offset angle α.

The image processing algorithm may be configured to use the rotated image and/or the rotated depth map to (i) improve an efficiency of the shadow mapping algorithm and/or (ii) reduce an amount of computation required to generate the one or more virtual shadows. The use of parallel virtual light beams in conjunction with the rotated image and/or the rotated depth map may improve an efficiency of the shadow mapping algorithm by permitting the shadow mapping algorithm to compute a shading or lighting effect for one or more pixels directly aligned with the parallel virtual light beams as a function of a distance between the one or more pixels within the region of interest and the one or more virtual light sources used to generate the parallel virtual light beams. In cases where the one or more pixels within the region of interest are directly aligned with the one or more parallel virtual light beams, the shadow mapping algorithm may be configured to compute a shading or lighting effect for each aligned pixel without needing to adjust such shading or lighting effects due to a positional offset of the virtual light beams relative to the one or more pixels within the region of interest. The use of one or more parallel virtual light beams in conjunction with the rotated image and/or the rotated depth map may further reduce an amount of computation required to generate the one or more virtual shadows by minimizing a number of calculations needed to determine a shading or lighting effect for one or more unaligned pixels within the region of interest (i.e., one or more pixels within the region of interest that are not aligned with the virtual light beams). The one or more pixels that are not aligned with the virtual light beams may comprise one or more pixels that are positioned such that the one or more virtual light beams intersect the image or the depth map (i) at a point that does not correspond to a pixel, or (ii) at a point that is offset from a location of a pixel.

In some cases, the shadow mapping algorithm may be configured to use the rotated image to compute shadow map values for an array of pixels arranged in one or more pixel columns that are aligned with the one or more virtual light beams, thereby reducing an amount of computation required to determine or approximate shadow map values for pixels that are not aligned with the one or more virtual light beams. In some cases, the shadow mapping algorithm may be configured to use the rotated image to process two or more columns of pixels in parallel when computing shadow map values for a plurality of pixels within the two or more columns of pixels, thereby improving an efficiency of the shadow mapping algorithm.

In some embodiments, the shadow mapping algorithm may be configured to generate the one or more virtual shadows in part using ray tracing. Ray tracing may involve tracing a path of light and simulating lighting effects for one or more pixels as the light encounters surfaces or features within the surgical scene. Ray tracing may involve extending rays of light from virtual light sources into a surgical scene and bouncing the rays of light off surfaces or features within the surgical scene. The rays of light may be reflected back towards the virtual light sources and may be used to approximate color values of one or more pixels within a portion of the surgical scene.

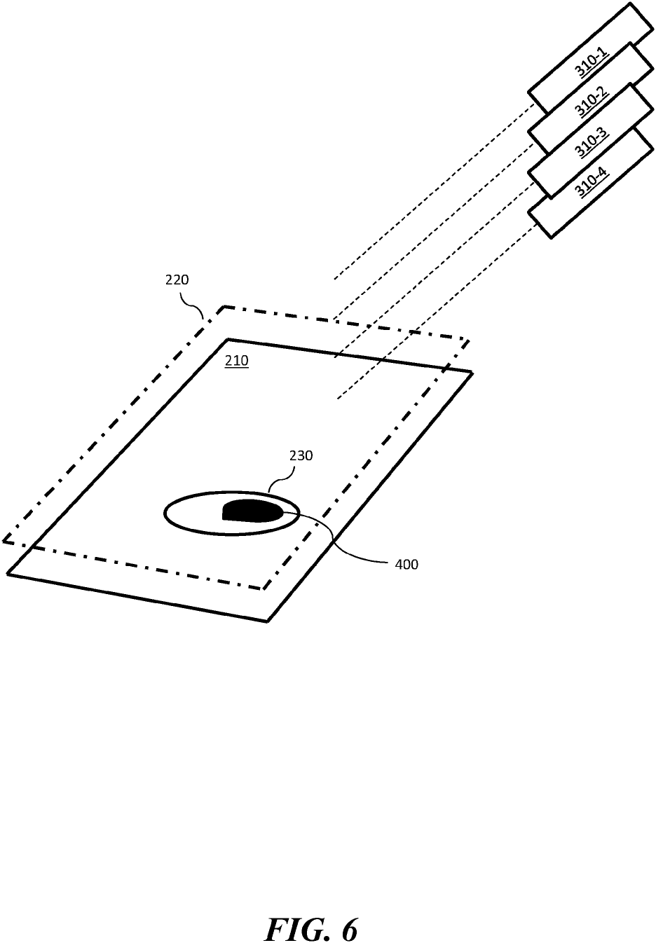

In some cases, the plurality of virtual light sources may be repositionable to generate the one or more virtual shadows in a different region of interest. FIG. 6 illustrates a plurality of virtual light sources that are repositioned to another position and/or orientation relative to the image 210 and the depth map 220. In some cases, the shadow mapping algorithm may be configured to generate one or more virtual shadows in a different region of interest. In other cases, the shadow mapping algorithm may be configured to generate one or more virtual shadows 400 in a different portion of a region of interest 230 previously identified by a surgeon or medical operator.

In another aspect, the present disclosure provides a method for enhancing depth perception to aid a surgical procedure. The method may comprise: (a) obtaining an image of a surgical scene and a depth map associated with the image, and (b) using an image processing algorithm to directly generate, based on the image and the depth map, one or more virtual shadows for enhancing depth perception in the image, without using or requiring computation of a three-dimensional (3D) representation of the surgical scene. As used herein, the term “three-dimensional representation” or “3D representation” of a surgical scene may correspond to a representation of the surgical scene that is distinct from a depth map. Such three-dimensional or 3D representation may comprise, for example, a point cloud or a mesh (e.g., a mesh of a surface of one or more objects, features, or tissue regions in the surgical scene), which comprises a different computational representation of the surgical scene than a depth map associated with or derived for the surgical scene. For instance, the depth map may comprise a two-dimensional (2D) array of data comprising depth information, whereas a three-dimensional representation may comprise a full 3D model, volume, or point cloud of the surgical scene. In the embodiments described herein, one or more virtual shadows may be computed directly from the 2D data structure of a depth map (which may comprise 3D cues or information embedded in the 2D data structure), and can be applied directly to the 2D data structure of an image, as opposed to other systems or methods that compute shadows in a full 3D model and thereafter re-project the shadows back onto a 2D image. In some cases, using a depth map to generate virtual shadows may be more computationally efficient than generating virtual shadows based on a full 3D model, volume, or point cloud of the surgical scene. The image may not or need not comprise a pre-operative image (e.g., a pre-operative scan such as a computed tomographic (CT) scan, a magnetic resonance imaging (MRI) scan, and an ultrasonography scan). The image may not or need not comprise a superimposed image comprising a pre-operative image or scan. The one or more virtual shadows may be generated without using or requiring computation of a three-dimensional (3D) representation of the surgical scene, such as a point cloud or a mesh associated with the surgical scene or an anatomical feature within the surgical scene.

The image processing algorithm may be configured to simulate a virtual light model comprising a plurality of virtual light sources. The plurality of virtual light sources may be configured to generate one or more virtual light beams that intersect the image of the surgical scene to generate one or more virtual shadows within a portion of the image.

The image processing algorithm may be configured to generate one or more virtual shadows by simulating a plurality of light sources. The plurality of light sources may be configured to generate one or more virtual light beams that intersect a portion of the image of the surgical scene or a modified image of the surgical scene. The one or more virtual light beams may generate one or more virtual shadows in one or more regions of interest within the image or the modified image after intersecting a portion of the image or the modified image. The modified image may be derived by rotating the image of the surgical scene to align two or more pixels of the image with one or more virtual light beams. As described above, the modified image may be used to optimize a computation or generation of the one or more virtual shadows within the image of the surgical scene.

In some embodiments, the one or more virtual light beams may be parallel. In some alternative embodiments, the one or more virtual light beams may be non-parallel. The one or more virtual light beams may be generated by a plurality of virtual light sources simulated within or near a computer-generated three-dimensional (3D) virtual scene that comprises an image of the surgical scene. The image of the surgical scene may be provided on a reference plane within the computer-generated 3D virtual scene, as described above. In some cases, the plurality of virtual light sources may be repositionable relative to the image or the depth map to generate one or more virtual shadows in a different portion of a previously identified region of interest. In other cases, the plurality of virtual light sources may be repositionable relative to the image or the depth map to generate one or more virtual shadows in different regions of interest.

The image processing algorithm may be configured to implement a shadow mapping algorithm to generate the one or more virtual shadows. The shadow mapping algorithm may be configured to compute shadow maps and generate one or more virtual shadows within an image of the surgical scene or a modified image of the surgical scene. The modified image may be derived by rotating the image of the surgical scene to align two or more pixels of the image with one or more virtual light beams.

As described above, the image processing algorithm may be configured to implement a shadow mapping algorithm. In some cases, the image processing algorithm may be configured to use a modified or rotated image of the surgical scene to (i) improve an efficiency of the shadow mapping algorithm and/or (ii) reduce an amount of computation required to generate the one or more virtual shadows within an image of the surgical scene.

In some embodiments, the shadow mapping algorithm may be configured to use the modified or rotated image to compute shadow map values for an array of pixels arranged in a plurality of pixel columns that are aligned with the one or more virtual light beams. Such a configuration may reduce an amount of computation required to generate shadow map values for pixels that are not aligned with the one or more virtual light beams. The shadow mapping algorithm may be further configured to use the modified image to process two or more columns of pixels in parallel when computing shadow map values for a plurality of pixels within the two or more columns of pixels, thereby improving an efficiency of the shadow mapping algorithm. As described elsewhere herein, shadow map values may comprise numerical values associated with a color, an opacity, a brightness, and/or a degree of shading of one or more pixels within an image or a rotated image of a surgical scene. The one or more pixels may correspond to a virtual shadow or a portion of a virtual shadow in a region of interest within the image or the rotated image of the surgical scene.

In some cases, the shadow mapping algorithm may be configured to generate the one or more virtual shadows in part using ray tracing. Ray tracing may involve tracing a path of light and simulating lighting effects for one or more pixels as the light encounters surfaces or features within the surgical scene. Ray tracing may involve extending rays of light from virtual light sources into a surgical scene and bouncing the rays of light off surfaces or features within the surgical scene. The rays of light may be reflected back towards the virtual light sources and may be used to approximate color values of one or more pixels within a portion of the surgical scene.

In another aspect, the present disclosure provides a system for enhancing depth perception to aid a surgical procedure. The system may comprise (a) a scope that is insertable into a body of a subject and (b) an imaging device optically coupled to the scope. The imaging device may be integrated with the scope. The scope may comprise a laparoscope, an endoscope, a borescope, a videoscope, or a fiberscope. The imaging device may be configured to (i) obtain one or more two-dimensional (2D) images of a surgical scene within the body of the subject and (ii) measure depth information or compute a topology of the surgical scene. The depth information and/or the topology of the surgical scene may be obtained using a time of flight (TOF) sensor. The TOF sensor may be integrated with the imaging device. The system may further comprise (c) an image processing module configured to use the one or more two-dimensional images and at least one of (i) the depth information or (ii) the topology of the surgical scene to directly generate one or more virtual shadows in the one or more two-dimensional (2D) images. The one or more virtual shadows may be generated without using or requiring computation of a three-dimensional (3D) representation of the surgical scene. The one or more two-dimensional (2D) images may not or need not comprise a pre-operative scan or a superimposed image comprising a pre-operative scan.

The image processing module may be configured to simulate a plurality of virtual light sources configured to generate one or more virtual light beams that intersect the one or more two-dimensional (2D) images of the surgical scene. The one or more virtual light beams may be parallel to one another.

In some cases, the image processing module may be configured to generate the one or more virtual shadows using a modified image of the surgical scene. The modified image may be derived by rotating the one or more two-dimensional (2D) images to align two or more pixels of the 2D images with the one or more virtual light beams. The one or more two-dimensional (2D) images may be rotated by a rotational angle. The rotational angle may be computed based on an angle formed between (i) a reference line comprising two or more pixels within the 2D images and (ii) a projection of the one or more virtual light beams onto a reference plane containing the 2D images. The rotational angle may be greater than 0 degrees and less than or equal to 360 degrees.

The image processing module may be configured to implement a shadow mapping algorithm to generate the one or more virtual shadows. As described elsewhere herein, the image processing module may be configured to use the modified image to (i) improve an efficiency of the shadow mapping algorithm and (ii) reduce an amount of computation required to generate the one or more virtual shadows.

In some cases, the plurality of virtual light sources simulated by the image processing module may be repositionable to generate one or more virtual shadows in a different portion of a region of interest within the one or more two-dimensional (2D) images of the surgical scene. In some cases, the plurality of virtual light sources simulated by the image processing module may be repositionable to generate one or more virtual shadows in a different region of interest within the one or more two-dimensional (2D) images of the surgical scene.

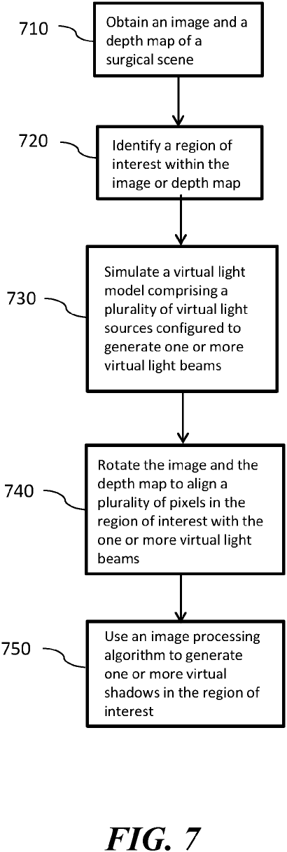

FIG. 7 illustrates an example of a method for enhancing depth perception in an image to aid a surgical procedure. The method may comprise: (a) using a scope and an imaging device to obtain an image of a surgical scene and a depth map associated with the image (710), (b) identifying a region of interest within the image or the depth map (720), (c) simulating a virtual light model comprising a plurality of virtual light sources configured to generate one or more virtual light beams that intersect the image of the surgical scene (730), (d) rotating the depth map and the image of the surgical scene by a rotational angle to align the plurality of pixels with the one or more virtual light beams (740), and (e) using an image processing algorithm to generate one or more virtual shadows for one or more portions of the region of interest based in part on the rotated image and the rotated depth map (750).

### Computer Systems

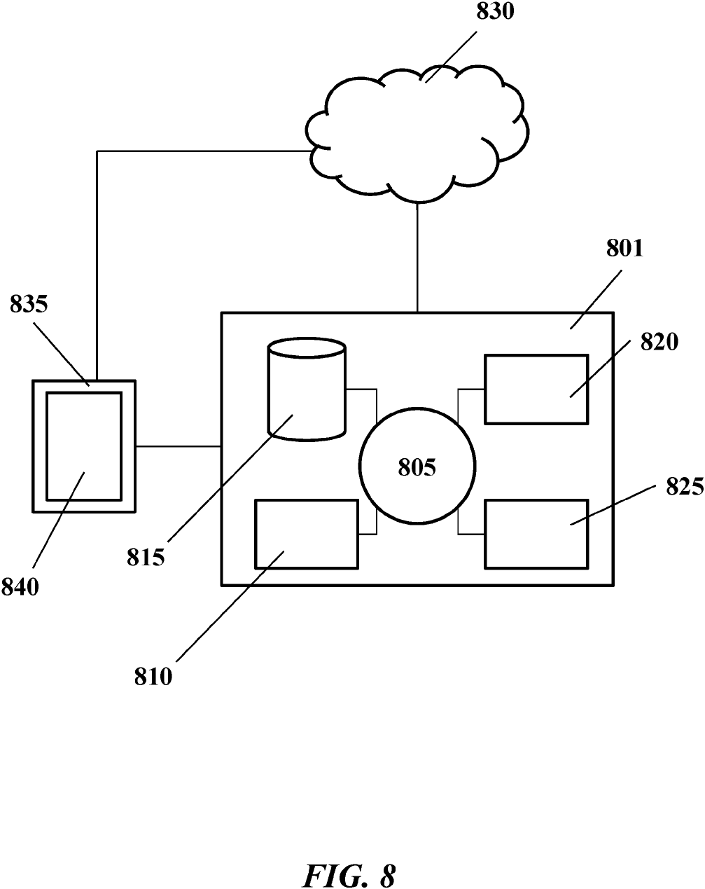

Another aspect of the present disclosure provides computer systems that are programmed or otherwise configured to implement methods of the disclosure. FIG. 8 shows a computer system 801 that is programmed or otherwise configured to implement a method for medical imaging. The computer system 801 may be configured to (a) use a scope and an imaging device to obtain an image of a surgical scene and a depth map associated with the image, (b) identify a region of interest within the image or the depth map, (c) simulate a virtual light model comprising a plurality of virtual light sources configured to generate one or more virtual light beams that intersect the image of the surgical scene, (d) rotate the depth map and the image of the surgical scene by a rotational angle to align the plurality of pixels with the one or more virtual light beams, and (e) use an image processing algorithm to generate one or more virtual shadows for one or more portions of the region of interest based in part on the rotated image and the rotated depth map. The computer system 801 can be an electronic device of a user or a computer system that is remotely located with respect to the electronic device. The electronic device can be a mobile electronic device.

The computer system 801 may include a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are located external to the computer system 801 (e.g., on a remote server that is in communication with the computer system 801 through an intranet or the Internet).

The computer system 801 can communicate with one or more remote computer systems through the network 830. For instance, the computer system 801 can communicate with a remote computer system of a user (e.g., a patient, a subject, a doctor, a medical operator, a surgical operator, a nurse, a surgeon, etc.). Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a portal for modifying a position and/or an orientation of one or more virtual light sources and identifying a region of interest in the image or the depth map of the surgical scene. In some cases, the portal may be used to render, view, monitor, and/or manipulate one or more images or depth maps obtained using the systems or methods disclosed herein. In other cases, the portal may be used to render, view, monitor, and/or manipulate one or more virtual shadows generated within a region of interest of one or more images or depth maps obtained using the systems or methods disclosed herein. The portal may be provided through an application programming interface (API). A user or entity can also interact with various elements in the portal via the UI. Examples of UI’s include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm may be configured to (a) use a scope and an imaging device to obtain an image of a surgical scene and a depth map associated with the image, (b) identify a region of interest within the image or the depth map, (c) simulate a virtual light model comprising a plurality of virtual light sources configured to generate one or more virtual light beams that intersect the image of the surgical scene, (d) rotate the depth map and the image of the surgical scene by a rotational angle to align the plurality of pixels with the one or more virtual light beams, and (e) use an image processing algorithm to generate one or more virtual shadows for one or more portions of the region of interest based in part on the rotated image and the rotated depth map. In some cases, the image processing algorithm may be configured to reposition the plurality of virtual light sources.

In another aspect, the present disclosure provides a method for enhancing medical images. The method may comprise: (a) obtaining an initial image of a surgical scene and a depth map associated with the initial image, and (b) adjusting a shading of one or more pixels of the initial image based at least in part on (i) a light intensity fall-off pattern and (ii) the depth map associated with the initial image of the surgical scene, to generate an updated image having adjusted shading. The updated image having the adjusted shading may provide enhanced depth perception to aid a surgical procedure at or near the surgical scene.

The initial image of the surgical scene may have an initial shading. The initial shading may comprise a variation in color, brightness, and/or shading within a portion of the surgical scene, relative to other regions within the surgical scene. The initial shading may comprise a variation in a level of darkness or a level of brightness within a portion of the surgical scene, relative to other regions within the surgical scene. The initial shading may correspond to one or more variations in lighting due to a geometry or a topology of the surgical scene. In some cases, the initial shading may correspond to one or more variations in lighting due to a configuration of a scope used to obtain the initial image. For example, the initial image may exhibit a radial shading gradient that is produced when a scope (e.g., a laparoscope) is used to obtain the initial image. The radial shading gradient may be a shading gradient that varies as a function of an inverse square of a distance from a center point of illumination. The radial shading gradient may be produced when a light is provided through the laparoscope to obtain one or more images of a surgical scene. The light intensity fall-off pattern may comprise a shading pattern that corresponds to the radial shading gradient produced when the surgical scene is imaged using a scope (e.g., a laparoscope).

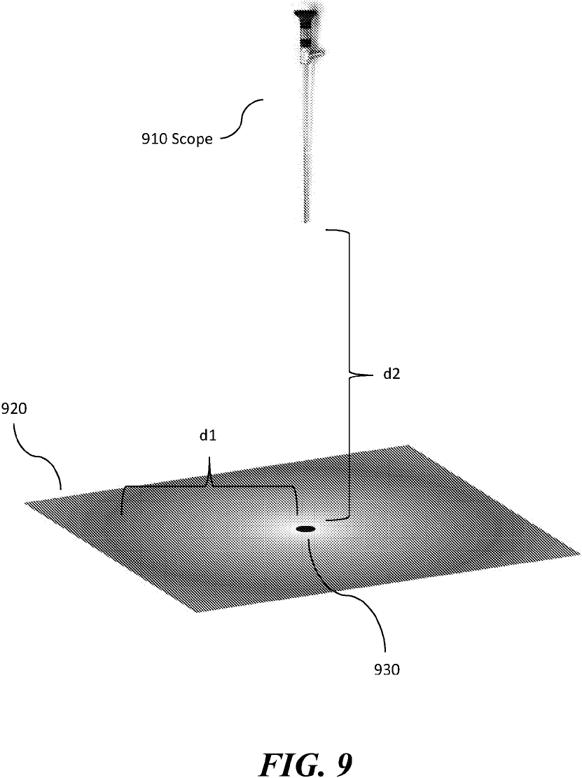

FIG. 9 illustrates a scope 910 used to obtain an initial image 920 of a surgical scene. The initial image of the surgical scene may be obtained by illuminating the surgical scene with light directed from an illumination source through the scope. The scope may comprise a laparoscope, an endoscope, a borescope, a videoscope, or a fiberscope. The initial image 920 may exhibit an initial shading. The initial shading may comprise a radial shading gradient. The radial shading gradient may correspond to a light intensity fall-off pattern that varies as a function of an inverse square of a distance d1 from a center point of illumination 930. The light intensity fall-off pattern may also vary as a function of a distance d2 from a tip of the scope 910 to the center point of illumination 930. The light intensity fall-off pattern may be a function of (i) a vertical distance from a tip of the scope to a center point of illumination within the surgical scene and (ii) a horizontal distance from the center point of illumination to the one or more pixels of the initial image.

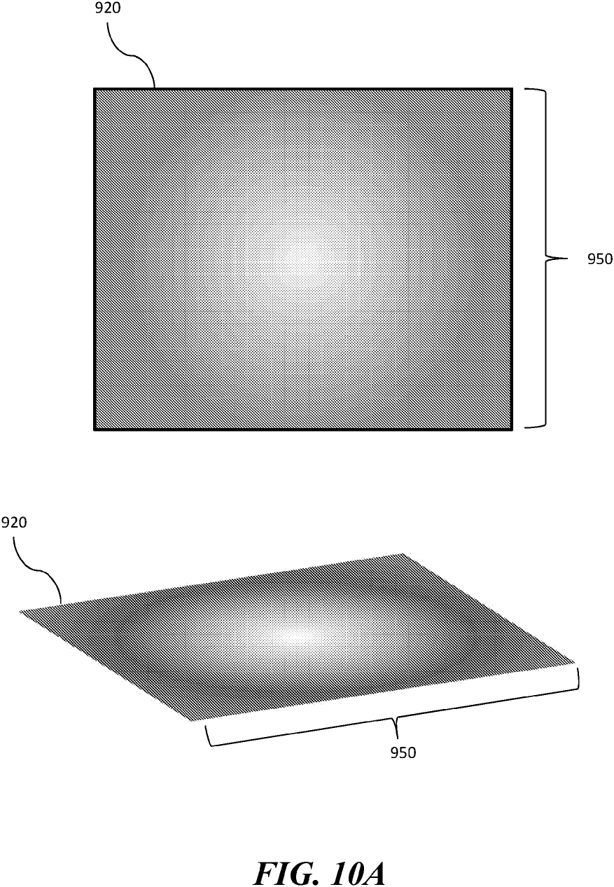

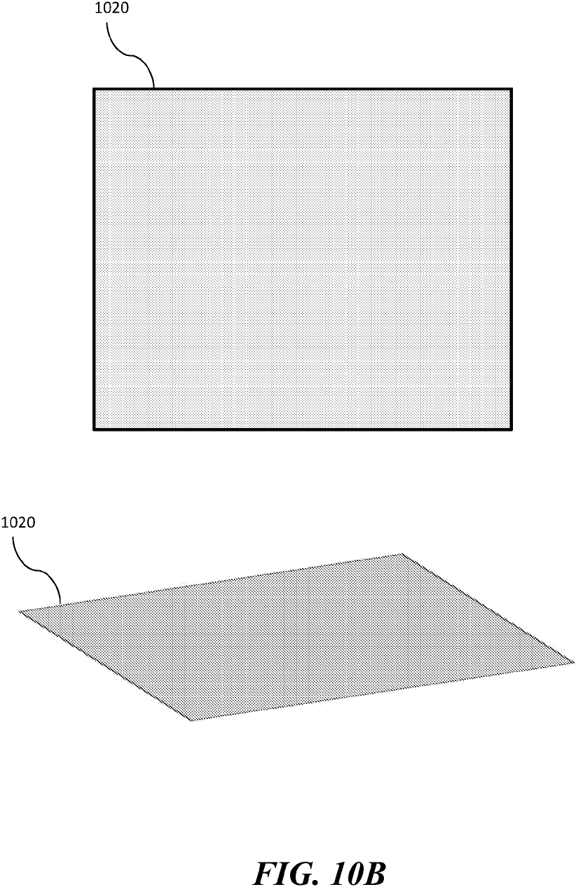

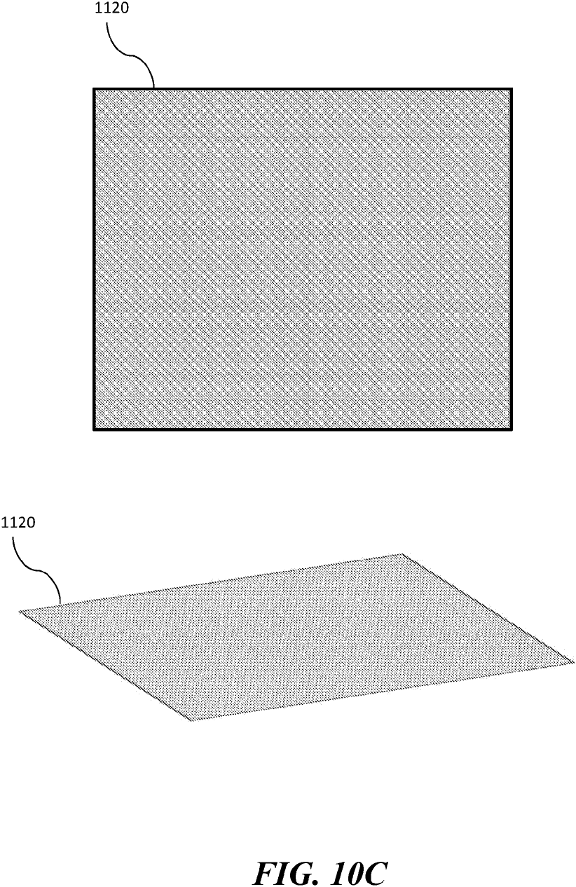

FIG. 10A illustrates an example of an initial image 920 of a surgical scene with a radial shading gradient 950. In some cases, the initial image 920 may further comprise a shading generated due to a topology of the surgical scene being imaged. The systems and methods disclosed herein may be used to adjust both a shading due to a topology of the surgical scene and a shading due to a light intensity fall-off pattern that is produced when an image of the surgical scene is obtained using a scope. Adjusting the shading of one or more pixels within the initial image may comprise modifying a color intensity, a brightness, or an opacity of the one or more pixels. In some cases, adjusting the shading may comprise removing (i) one or more shading effects due to a topology of the surgical scene and/or (ii) one or more shading effects due to a light intensity fall-off pattern that is produced when an image of the surgical scene is obtained using a scope. The removal of such shading effects may produce an unshaded image 1020 as shown in FIG. 10B.

As described above, adjusting the shading of one or more pixels within an initial image of a surgical scene may comprise removing one or more shading effects due to a topology of the surgical scene. In such cases, adjusting the shading of the one or more pixels may involve (i) computing surface normal information from a depth map associated with the initial image and (ii) using the surface normal information to remove existing shading in the initial image due to a topology of the surgical scene, thereby creating an unshaded image. The surface normal information may comprise information associated with and/or characterizing a normal vector at every point or pixel within the surgical scene. In some cases, adjusting the shading of the one or more pixels may involve calibrating an illumination of the initial image at a plurality of depths to remove one or more illumination or shading effects generated within the initial image by the illumination source. The removal of the one or more illumination or shading effects may produce an unshaded image. The unshaded image may comprise an image of the surgical scene with a uniform color intensity. In some cases, adjusting the shading of the one or more pixels based on the depth map may comprise modifying the shading of the one or more pixels based at least in part on a relative distance or a relative orientation of one or more features associated with the one or more pixels, in relation to portion of the scope (e.g., a tip of the scope).

After a removal of one or more existing shading effects due to a topology of the surgical scene and/or a light intensity fall-off pattern, an additional new shading based on the surface normal information may be applied to the unshaded image to generate an updated image having adjusted shading. FIG. 10C illustrates an updated image 1120 with an additional new shading. A new adjusted shading based on surface normal information derived from the depth map may be applied to the unshaded image to generate the updated image having the adjusted shading. The adjusted shading may provide a more uniform scene depiction across the updated image, relative to the initial image. The depth map used to derive the surface normal information may be obtained using a time of flight (TOF) sensor or a stereoscopic camera.

In some cases, the initial image and the updated image may comprise at least a portion of a laser speckle contrast image. In some cases, the initial image and the updated image may comprise a physiological visualization of a perfusion pattern obtained from a laser speckle contrast image, or a tissue classification obtained from hyperspectral imaging.

In some embodiments, the method may further comprise simulating one or more virtual light sources and modifying a shading of one or more pixels of the initial image or the updated image based on a relative position or a relative orientation of the one or more virtual light sources in relation to the one or more pixels within the initial image or the updated image. The one or more virtual light sources may be repositionable relative to the initial image or the updated image of the surgical scene.

In some embodiments, the method may further comprise: using the initial image and the updated image to compute a blood flow velocity through one or more blood vessels within the surgical scene, based in part on (i) a size of the one or more blood vessels and (ii) a concentration of blood within the one or more blood vessels. The size of the one or more blood vessels and the concentration of blood within the one or more blood vessels may be determined in part based on the initial image, the updated image, or a comparison of the initial image against the updated image.

In another aspect, the present disclosure provides a system for enhancing medical images. The system may comprise an image processing module comprising one or more processors that, upon execution of a set of instructions stored in memory, are configured to: (a) obtain an initial image of a surgical scene and a depth map associated with the initial image and (b) adjust a shading of one or more pixels of the initial image based on (i) a light intensity fall-off pattern and (ii) the depth map, to generate an updated image having adjusted shading. As described elsewhere herein, adjusting the shading of the one or more pixels may comprise using surface normal information obtained from the depth map to generate an unshaded image of the surgical scene from the initial image. The unshaded image may comprise an image of the surgical scene with a uniform color intensity. An additional shading based on the surface normal information may be applied to the unshaded image to generate an updated image with adjusted shading. The updated image with the adjusted shading may provide a more uniform scene depiction with a uniform brightness across the updated image.

In another aspect, the present disclosure provides a method for augmented medical imaging. The method may comprise (a) obtaining an initial scope image of a deformable tissue surface, an initial depth map corresponding to the initial scope image, and an initial pre-operative image of the deformable tissue surface. The initial scope image may comprise an image of a deformable tissue surface that is obtained using a scope. The deformable tissue surface may comprise a portion of a critical structure within a subject’s body. The scope may comprise a laparoscope, an endoscope, a borescope, a videoscope, or a fiberscope. The initial pre-operative image may comprise a computed tomographic (CT) scan, a magnetic resonance imaging (MRI) scan, and/or an ultrasonography scan. The deformable tissue surface may correspond to a portion of a tissue surface that may be deformed in response to an external force exerted on the tissue surface by a medical instrument or a medical operator (e.g., a surgeon). The tissue surface may correspond to a portion of a surface of an epithelial tissue, a connective tissue, a muscle tissue (e.g., skeletal muscle tissue, smooth muscle tissue, and/or cardiac muscle tissue), and/or a nerve tissue. Examples of the deformable tissue surface may include, but are not limited to, a tissue surface of a thyroid gland, adrenal gland, mammary gland, prostate gland, testicle, trachea, superior vena cava, interior vena cava, lung, liver, gallbladder, kidney, ureter, appendix, bladder, urethra, heart, esophagus, diaphragm, aorta, spleen, stomach, pancreas, small intestine, large intestine, rectum, vagina, ovary, bone, thymus, skin, adipose, eye, brain, fetus, arteries, veins, nerves, ureter, bile duct, healthy tissue, and/or diseased tissue.

The method may further comprise (b) overlaying or superimposing the initial pre-operative image onto the initial scope image, or vice versa, to generate an initial superimposed image. In some cases, the initial pre-operative image may be overlaid onto the initial scope image or a portion thereof. In other cases, the initial scope image may be overlaid onto the initial pre-operative image or a portion thereof.

The method may further comprise (c) obtaining a subsequent scope image of the deformable tissue surface and an updated depth map corresponding to the subsequent scope image. The initial scope image may correspond to an image of the deformable tissue surface in an undeformed state. The subsequent scope image may correspond to an image of the deformable tissue surface in a deformed state.

The method may further comprise (d) computing a deformation delta based at least in part on the initial depth map and the updated depth map. The deformation delta may be computed in part based on a difference between one or more values of the initial depth map and one or more values of the updated depth map. The deformation delta may correspond to a difference between a topology of the deformable tissue surface in an undeformed state and a topology of the deformable tissue surface in a deformed state.

The method may further comprise (e) using the deformation delta to generate a modified pre-operative image from the initial pre-operative image. The modified pre-operative image may comprise a representation of the initial pre-operative that is modified to account for a change in a topology of the deformable tissue surface after the deformable tissue surface is deformed by an external force.

The method may further comprise (f) overlaying the modified pre-operative image onto the subsequent scope image, or vice versa, to generate an updated superimposed image. The updated superimposed image may correspond to the deformable tissue surface in a deformed state. The updated superimposed image may provide a visualization of the deformable tissue surface in the deformed state and may include one or more visual features provided by a pre-operative image.

In some cases, the steps of (c) - (f) may be performed substantially in real-time for a series of subsequent scope images. The series of subsequent scope images may comprise one or more scope images taken while the deformable tissue surface undergoes a deformation.

In another aspect, the present disclosure provides a method for augmented medical imaging. The method may comprise: (a) obtaining an initial scope image of a deformable tissue surface, an initial depth map of the deformable tissue surface, and an initial pre-operative image of the deformable tissue surface. The method may further comprise: (b) overlaying the initial pre-operative image onto the initial scope image, or vice versa, to generate an initial superimposed image. The method may further comprise: (c) obtaining a subsequent scope image of the deformable tissue surface and an updated depth map of the deformable tissue surface. The method may further comprise: (d) computing a deformation delta based at least in part on the initial depth map and the updated depth map. The method may further comprise: (e) using the deformation delta to generate an updated superimposed image from the initial superimposed image. The updated superimposed image may correspond to the deformable tissue surface in a deformed state. The updated superimposed image may provide a visualization of the deformable tissue surface in the deformed state and may include one or more visual features provided by a pre-operative image.

In some cases, the steps of (c) - (e) may be performed substantially in real-time for a series of subsequent scope images. The series of subsequent scope images may comprise one or more scope images taken while the deformable tissue surface undergoes a deformation. As described above, the initial scope image may correspond to an image of the deformable tissue surface in an undeformed state. The subsequent scope image may correspond to an image of the deformable tissue surface in a deformed state.

In another aspect, the present disclosure provides a method for augmented medical imaging. The method may comprise: (a) obtaining an initial scope image of a deformable tissue surface and an initial pre-operative image of the deformable tissue surface and (b) generating an initial depth map of the deformable tissue surface based at least in part on the initial scope image. The initial pre-operative image may comprise a computed tomographic (CT) scan, a magnetic resonance imaging (MRI) scan, and/or an ultrasonography scan.

The method may further comprise: (c) identifying a first set of target points on the initial scope image and the initial pre-operative image. The first set of target points may comprise at least one similar feature in both the initial scope image and the initial pre-operative image. The at least one similar feature may comprise a portion of a region of interest within the initial scope image and/or the initial pre-operative image.

The method may further comprise: (d) using the first set of target points to register and overlay the initial pre-operative image onto the initial scope image, or vice versa. In some cases, the first set of target points may be used to overlay the initial pre-operative image onto the initial scope image. In other cases, the first set of target points may be used to overlay the initial scope image onto the initial pre-operative image.

The method may further comprise: (e) obtaining a subsequent scope image of the deformable tissue surface. As described above, the subsequent scope image may correspond to an image of the deformable tissue surface in a deformed state. The initial scope image may correspond to an image of the deformable tissue surface in an undeformed state.

The method may further comprise: (f) generating an updated depth map of the deformable tissue surface based at least in part on the subsequent scope image and (g) computing a deformation delta based at least in part on the initial depth map and the updated depth map. The deformation delta may represent a difference between one or more values of the initial depth map and one or more values of the updated depth map. The one or more values of the initial depth map and the one or more values of the updated depth map may be associated with a similar feature within the initial scope image and the subsequent scope image. The deformation delta may correspond to a difference in a topology of the deformable tissue surface in an undeformed state and a topology of the deformable tissue surface in a deformed state.

The method may further comprise: (h) using the deformation delta to generate a modified pre-operative image from the initial pre-operative image and to identify a second set of target points in the subsequent scope image and the modified pre-operative image. The second set of target points may comprise the at least one similar feature associated with the first set of target points. In some cases, the first set of target points and the second set of target points may correspond to at least a portion of a blood perfusion pattern. In other cases, the first set of target points and the second set of target points may correspond to at least a portion of a perfusion pattern associated with a bodily fluid of a subject (e.g., blood, lymph, tissue fluid, milk, saliva, semen, bile, etc.).

The method may further comprise: (i) using the second set of target points to register and overlay the modified pre-operative image onto the subsequent scope image, or vice versa. In some cases, the steps of (e) - (i) may be performed substantially in real-time for a series of subsequent scope images. The series of subsequent scope images may comprise one or more scope images taken while the deformable tissue surface undergoes a deformation.

In any of the embodiments disclosed herein, the initial scope image and the subsequent scope image may be obtained using an imaging device and a scope. The scope may comprise a laparoscope, an endoscope, a borescope, a videoscope, or a fiberscope. In some cases, the imaging device may be integrated with the scope.

In some cases, the initial depth map and the updated depth map may be obtained using a time of flight (TOF) sensor. The TOF sensor may be integrated with the imaging device.

In any of the embodiments described herein, the initial scope image may correspond to an image of the deformable tissue surface in an undeformed state. In any of the embodiments described herein, the subsequent scope image may correspond to an image of the deformable tissue surface in a deformed state.

In another aspect, the present disclosure provides a method for augmented medical imaging. The method may comprise: (a) obtaining an initial scope image of a deformable tissue surface and an initial pre-operative image of the deformable tissue surface; (b) generating an initial depth map of the deformable tissue surface from the initial scope image; and (c) identifying one or more points of interest on the initial scope image and the initial pre-operative image. The one or more points of interest may comprise at least one similar feature in both the initial scope image and the initial pre-operative image. The method may further comprise: (d) using the one or more points of interest to register and overlay the initial pre-operative image onto the initial scope image, or vice versa, thereby generating an overlaid image.

The method may further comprise: (e) obtaining a subsequent scope image of the deformable tissue surface; (f) generating an updated depth map of the deformable tissue surface using at least the subsequent scope image; and (g) computing a deformation delta based at least in part on the initial depth map and the updated depth map. The method may further comprise: (h) using the deformation delta to generate an updated overlaid image based at least in part on the overlaid image. The updated overlaid image may correspond to the deformable tissue surface in a deformed state.

In any of the embodiments described herein, the initial scope image and the subsequent scope image may be obtained using a scope. The scope may comprise a laparoscope, an endoscope, a borescope, a videoscope, and/or a fiberscope.

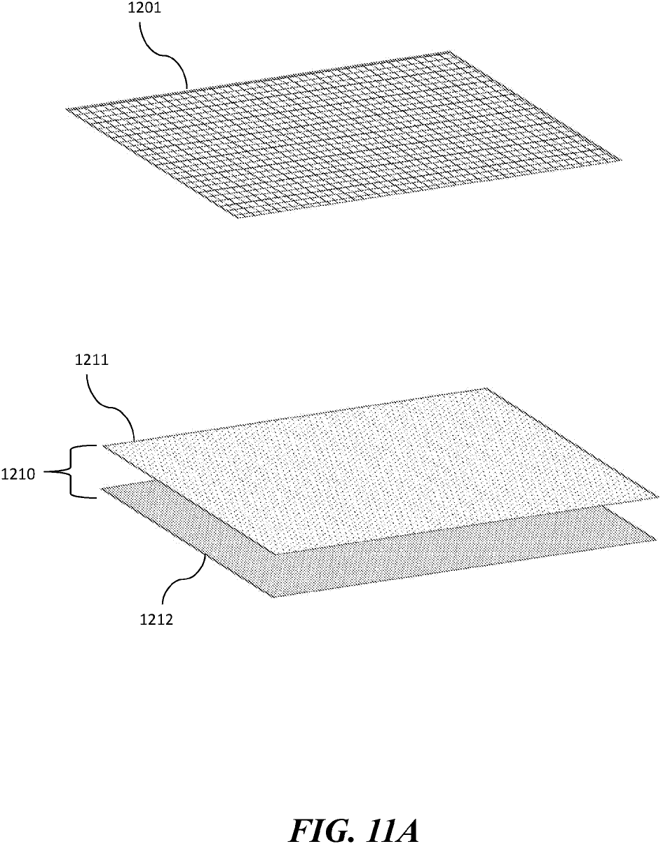

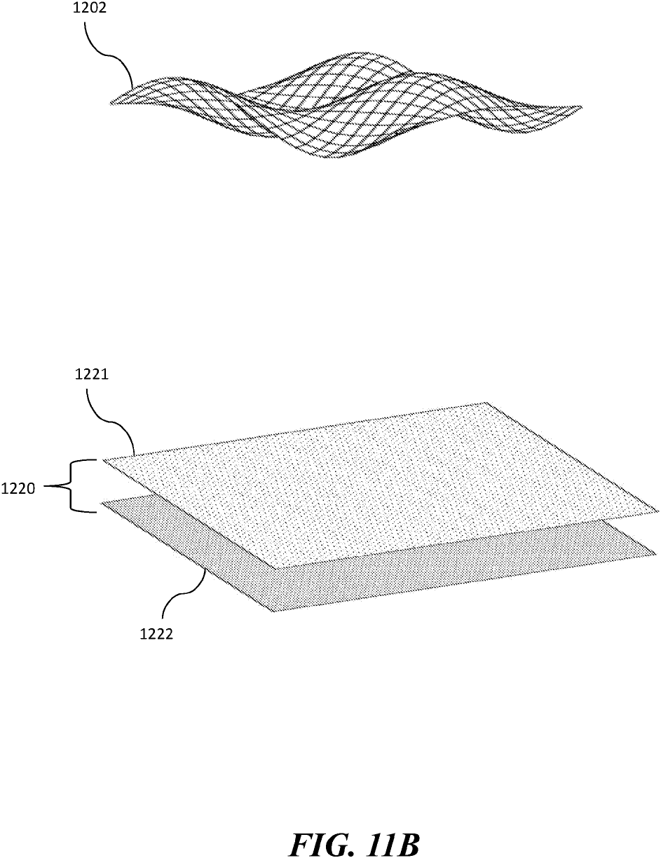

FIG. 11A illustrates a deformable tissue surface 1201 in an undeformed state. An initial superimposed image 1210 may be generated by overlaying an initial pre-operative image 1211 onto an initial scope image 1212. The initial scope image 1212 and the initial pre-operative image 1211 may correspond to the deformable tissue surface 1201 in an undeformed state.

FIG. 11B illustrates the deformable tissue surface 1202 in a deformed state. A deformation delta may be computed by comparing depth map values associated with the deformable tissue surface in a deformed state 1202 against depth map values associated with the deformable tissue surface in an undeformed state 1201, as shown in FIG. 11A. The depth map values associated with the deformable tissue surface in the deformed state 1202 may be derived from a subsequent scope image 1222 of the deformable tissue surface after the deformable tissue surface undergoes a physical deformation due to an external force exerted on the tissue surface by a medical instrument or a medical operator. In some cases, an updated superimposed image 1220 may be generated by modifying the initial superimposed image 1210 (shown in FIG. 11A) based on the deformation delta computed from the comparison of the depth map values for the deformable tissue surface in a deformed state 1202 against the depth map values for the deformable tissue surface in an undeformed state 1201. In other cases, a modified pre-operative image 1221 may be generated from the initial pre-operative image 1211 of FIG. 11A using the deformation delta. In such cases, the updated superimposed image 1220 may be generated by overlaying the modified pre-operative image 1221 onto the subsequent scope image 1222.

In another aspect, the present disclosure provides a system for augmented medical imaging. The system may comprise (a) an imaging device configured to obtain an initial scope image of a deformable tissue surface and a subsequent scope image of the deformable tissue surface. The system may further comprise (b) a depth sensor configured to generate an initial depth map of the deformable tissue surface using the initial scope image and an updated depth map of the deformable tissue surface using the subsequent scope image. The system may further comprise (c) an image processing module. The image processing module may be configured to overlay an initial pre-operative image of the deformable tissue surface onto the initial scope image, or vice versa, based at least in part on a first set of target points identified in the initial scope image and the initial pre-operative image. The first set of target points may comprise at least one similar feature in both the initial scope image and the initial pre-operative image. As described elsewhere herein, the initial pre-operative image may comprise a pre-operative scan such as a computed tomographic (CT) scan, a magnetic resonance imaging (MRI) scan, and/or an ultrasonography scan, for example. The image processing module may be configured to compute a deformation delta based at least in part on the initial depth map and the updated depth map. The image processing module may be configured to use the deformation delta to (i) generate a modified pre-operative image from the initial pre-operative image and (ii) overlay the modified pre-operative image onto the subsequent scope image, or vice versa, based at least in part on a second set of target points in the subsequent scope image and the modified pre-operative image. The second set of target points may comprise the at least one similar feature associated with the first set of target points. As described elsewhere herein, the initial scope image may comprise an image of the deformable tissue surface in an undeformed state, and the subsequent scope image may comprise an image of the deformable tissue surface in a deformed state.

In any of the embodiments described herein, an imaging device may be configured to obtain the initial scope image and the subsequent scope image via a scope. The imaging device may comprise a camera, a video camera, a three-dimensional (3D) depth camera, a stereo camera, a depth camera, a Red Green Blue Depth (RGB-D) camera, a time-of-flight (TOF) camera, an infrared camera, a charge coupled device (CCD) image sensor, or a complementary metal oxide semiconductor (CMOS) image sensor. The scope may comprise a laparoscope, an endoscope, a borescope, a videoscope, and/or a fiberscope. In some cases, the imaging device may be integrated with the scope.

As described above, the system may comprise a depth sensor configured to generate one or more depth maps of the deformable tissue surface. The depth sensor may comprise a time of flight (TOF) sensor or a stereoscopic camera.

In another aspect, the present disclosure provides a system for simulating binocular vision. The system may comprise an image processing unit comprising one or more processors configured to generate a plurality of images of a surgical scene based on imaging data obtained using one or more imaging sensors and/or one or more depth sensors. The plurality of images of the surgical scene may comprise a pair of images associated with a surgical scene. A first image of the pair of images may be captured, obtained, or generated using a first imaging sensor or a first virtual camera provided in a first location. A second image of the pair of images may be captured, obtained, or generated using a second imaging sensor or a second virtual camera provided in a second location. The first location and the second location may be different. The first imaging sensor or virtual camera may be provided at a first orientation relative to a region of interest in the surgical scene. The second imaging sensor or virtual camera may be provided at a second orientation relative to the region of interest in the surgical scene. The first orientation and the second orientation may be different.

In some cases, the image processing unit may be configured to simulate binocular vision using at least one image of a surgical scene and depth information associated with the at least one image of the surgical scene. The image processing unit may be configured to generate a first image based on the at least one image of the surgical scene and/or the depth information. The image processing unit may be further configured to generate a second image based on the at least one image of the surgical scene and/or the depth information. The second image may be altered relative to the first image such that the second image provides a modified view of the surgical scene that shows one or more features in the surgical scene from a different position and/or orientation compared to the first image. The second image may be spatially shifted relative to the first image. Such spatial shift may correspond to a separation distance between the imaging sensor or virtual camera used to obtain the first image and the imaging sensor or virtual camera used to obtain the second image. In some cases, the separation distance may correspond to a pupillary distance between the centers of the pupils of an operator viewing the images. The spatial shift may be adjusted based on one or more physical characteristics or features of the operator.

In some embodiments, the second image may be generated by (i) determining or estimating a position and/or orientation of the imaging sensor used to capture or generate the first image relative to the surgical scene, and (ii) simulating a virtual camera that provides a view of the surgical scene from a position and/or viewing angle that is different than that of the imaging sensor used to capture the first image. The second image may be generated based at least in part on depth information obtaining using any of the depth sensors described elsewhere herein. In some alternative embodiments, the first image and the second image may be obtained based on a movement of a same imaging sensor or virtual camera relative to the surgical scene or an image of the surgical scene. In any case, the first image and the second case may be viewed together (e.g., as a pair of corresponding left and right images of the surgical scene) to provide a simulated three-dimensional image or view of the surgical scene. Such simulated three-dimensional image or view of the surgical scene may be produced based on a parallax effect.

The first image and the second image used to provide the simulated three-dimensional image or view of the surgical scene may be displayed to an operator (e.g., a doctor or a surgeon) or a medical worker (e.g., a medical assistant) in order to enhance three-dimensional depth perception during a surgical procedure. The first image and the second image may be viewed by the operator or medical worker separately and individually. Alternatively, the first image and the second image may be viewed by the operator or medical worker in combination and/or simultaneously. In some cases, the first image and the second image may be provided to the operator via a display (e.g., a light field display or a monitor) or one or more interfaces for viewing images or videos in general (e.g., video goggles). The one or more interfaces may permit the operator or medical worker to view the first image, the second image, and/or both the first and second images, and to switch between any of these views as desired.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing one or more aspects of the present disclosure. It is therefore contemplated that the present disclosure shall also cover any such alternatives, modifications, variations or equivalents. It is intended that the following claims define the scope of the present disclosure and that the methods and structures within the scope of these claims and their equivalents be covered thereby.

What is claimed is:

1. A method for enhancing depth perception to aid a surgical procedure, the method comprising:
(a) obtaining an image of a surgical scene and a depth map associated with the image, wherein the image does not comprise a pre-operative image; and
(b) using an image processing algorithm to directly generate, based in part on the image and the depth map, one or more virtual shadows for enhancing depth perception in the image, without using or requiring a computation of a three-dimensional (3D) representation of the surgical scene.

2. The method of claim 1, wherein the image processing algorithm is configured to simulate a virtual light model comprising a plurality of virtual light sources, wherein the plurality of virtual light sources is configured to generate one or more virtual light beams that intersect the image of the surgical scene to generate one or more virtual shadows within a portion of the image.

3. The method of claim 1, wherein the image does not comprise a superimposed image.

4. The method of claim 1, wherein the pre-operative image comprises a pre-operative scan selected from the group consisting of a computed tomographic (CT) scan, a magnetic resonance imaging (MRI) scan, and an ultrasonography scan.

5. The method of claim 1, wherein the image processing algorithm is configured to implement a shadow mapping algorithm to generate the one or more virtual shadows.

6. The method of claim 5, wherein the shadow mapping algorithm is configured to generate the one or more virtual shadows using a modified image of the surgical scene, which modified image is derived by rotating the image of the surgical scene to align two or more pixels of the image with one or more virtual light beams.

7. The method of claim 6, wherein the shadow mapping algorithm is configured to use the modified image to compute shadow map values for an array of pixels arranged in one or more-pixel columns that are aligned with the one or more virtual light beams, thereby reducing an amount of computation required to generate shadow map values for pixels that are not aligned with the one or more virtual light beams.

8. The method of claim 6, wherein the shadow mapping algorithm is configured to use the modified image to process two or more columns of pixels in parallel when computing shadow map values for a plurality of pixels within the two or more columns of pixels, thereby improving an efficiency of the shadow mapping algorithm.

9. The method of claim 5, wherein the shadow mapping algorithm is configured to generate the one or more virtual shadows in part using ray tracing.

10. The method of claim 6, wherein the one or more virtual light beams are parallel.

11. The method of claim 1, wherein the three-dimensional (3D) representation comprises a 3D data array or data structure, a 3D volume, a point cloud, or a mesh associated with the surgical scene or an anatomical feature within the surgical scene.

12. The method of claim 6, wherein the one or more virtual light beams are generated using a plurality of virtual light sources simulated within or near the surgical scene.

13. The method of claim 12, wherein the plurality of virtual light sources are repositionable to generate the one or more virtual shadows in different regions of interest.

14-102. (canceled)

103. The method of claim 1, further comprising using a scope and an imaging device optically connected to the scope for obtaining the image.

104. The method of claim 103, wherein the scope is insertable into a body of a subject.

105. The method of claim 1, further comprising:
(a) identifying a region of interest within the image or the depth map, wherein the region of interest comprises a plurality of pixels;
(b) simulating a virtual light model, wherein the virtual light model comprises a plurality of virtual light sources configured to generate one or more virtual light beams that intersect the image of the surgical scene; and
(c) rotating the depth map and the image of the surgical scene by a rotational angle to align the plurality of pixels with the one or more virtual light beams;
wherein, the image processing algorithm generates the one or more virtual shadows for one or more portions of the region of interest, based in part on the rotated image and the rotated depth map.

106. The method of claim 105, wherein the image processing algorithm is configured to implement a shadow mapping algorithm to generate the one or more virtual shadows, and wherein the shadow mapping algorithm is configured to compute one or more shadow maps in part based on (i) a position and an orientation of the one or more virtual light sources relative to a portion of the region of interest and (ii) a comparison of depth values for two or more pixels within the portion of the region of interest.

107. The method of claim 106, wherein the two or more pixels comprise two or more adjacent pixels within a row or column of pixels.

108. The method of claim 1, wherein obtaining the depth map comprises using one or more depth sensors.

109. The method of claim 108, wherein the one or more depth sensors comprise a time-of-flight sensor or RGB-D sensor.
